(19) United States
(12) Patent Application Publication
(10) Pub. No.: US 2026/0306878 A1
(43) Pub. Date: Oct. 8, 2026

(54) SYSTEM AND METHOD FOR MULTI-TARGET SINGLE-ISOCENTER RADIATION THERAPY QUALITY ASSURANCE

(71) Applicant: Aktina Corp., Congers, NY (US)

(72) Inventors: Nicholas G. Zacharopoulos, West Nyack, NY (US); Thomas Cuthbert, Congers, NY (US)

(73) Assignee: Aktina Corp., Congers, NY (US)

(21) Appl. No.: 19/636,277

(22) Filed: Apr. 1, 2026

Related U.S. Application Data
(60) Provisional application No. 63/782,957, filed on Apr. 3, 2025.

(51) Int. Cl. A61N 5/10

(52) U.S. Cl. CPC A61N 5/1075; A61N 5/1045; A61N 5/1081; A61N 2005/1076

(57) ABSTRACT

Systems and methods for quality assurance (QA) in single-isocenter, multi-target (SIMT) radiation therapy. The QA may include measuring an actual position of an off-isocenter radiation opaque marker of a phantom, using the actual marker position to compute a beam's eye view marker position that is a projected position of the off-isocenter marker in an expected field contour of an expected radiation beam, and acquiring a 2D radiation transmission image indicative of a radiation field of a generated radiation beam after passing through the off-isocenter marker. The QA may include aligning the beam's eye view marker position with a centroid of a measured marker contour in the 2D radiation transmission image at a common marker reference point and determining a geometric error that is a 2D distance between a centroid of the expected field contour and a centroid of a measured field contour in the 2D radiation transmission image.

### CROSS-REFERENCE TO RELATED APPLICATION

The present application claims the benefit of priority to U.S. Provisional Patent Application Ser. No. 63/782,957, filed on Apr. 3, 2025, which is incorporated herein by reference in its entirety.

### BACKGROUND

### Field of Invention

The present invention relates generally to radiation therapy quality assurance. More particularly, aspects of the present invention relate to methods and systems for performing geometric accuracy testing for single-isocenter, multi-target (SIMT) radiation therapy treatments delivered by a medical linear accelerator (LINAC).

### Discussion of the Background

### 1.1. Medical Linear Accelerators

Radiation therapy is a type of cancer treatment that uses beams of intense high energy radiation beams to kill cancer cells. FIGS. 1-3 illustrates a medical linear accelerator (LINAC) 100 including a gantry 102 and a couch 106. As shown in FIG. 4, the gantry 102 includes a collimator head 104 that defines a field of a radiation beam 412 generated by the LINAC 100. During treatment, the gantry 102 of the LINAC 100 delivers the radiation beams to a precise location within a patient (not shown) supported on the couch 106. To customize a radiation treatment plan, the gantry 102 may rotate about the patient, and the couch 106 may changes the patient's orientation relative a plane of rotation of the gantry 102.

As shown in FIG. 2, the gantry 102 may be capable of rotating a full 360° around the patient so as to optimize the entry of the radiation beam into the patient. As shown in FIG. 2, the gantry 102 may rotate about a gantry axis of rotation 208. As shown in FIG. 3, the couch 106 may rotate about a couch axis of rotation 310. This allows the patient's orientation to be adjusted relative to the plane of rotation of the gantry 102 so as to further optimize the entry of the radiation beam 412 into the patient.

In an ideal LINAC 100, the radiation beam would remain focused on a fixed point in space (i.e., on a tumor in the patient) as the gantry 102 rotates. Similarly, the ideal LINAC 100 would keep the tumor fixed within the radiation beam as the patient moves due to rotation of the couch 106. The deviation between the center of the radiation beam and the center of the tumor is referred to as the geometric error. The LINAC isocenter is defined as the point in space that minimizes the maximum geometric error across all gantry and couch rotations.

As shown in FIG. 4, the LINAC 100 may have an imaging device 416 (e.g., an electronic portal imaging device (EPID)), which is used to verify the patients position before a treatment by measuring the x-ray intensity transmitted through a patient from a radiation port during a treatment session.

### 1.2 The Winston-Lutz Test

An existing method for analyzing isocenter is the Winston-Lutz (WL) test. The WL test includes two main steps: (1) measure EPID x-ray transmission images of a radiation-opaque marker positioned at (or near) isocenter with different gantry and couch rotations and (2) analyze the images to determine the LINAC geometric errors for different gantry and couch angle combinations.

The setup shown in FIG. 4 is typically used to accomplish the first step. In FIG. 4, the LINAC 100 is delivering a radiation beam 412 through a radiation opaque marker 414 that is placed at isocenter. The imaging device 416 acquires a two-dimensional transmission image of the radiation beam 412 as it passes through the marker 414.

A detailed view of a marker assembly 500 including the radiation opaque marker 414 is shown in FIG. 5. In FIG. 5, a high density spherical marker 414 is connected to a low density support rod 502 that then connects to a base 504. The base 504 is set on a top of the couch 106 so that the marker 414 can be positioned at the isocenter of the LINAC 100.

FIG. 6 shows a typical image 600 (e.g., an EPID image) through the marker 414. In FIG. 6, the dark square region 602 is created by a square radiation field of the radiation beam 412 exposing the imaging device 410, and the lighter inner circular shape 604 is created by the shadow of the radiation opaque marker 414 that is located within the radiation field.

Measuring the LINAC's geometric errors involves rotating the gantry 102 through a set of fixed gantry angles while the couch 106 remains at 0° and acquiring an image (e.g., an EPID image) for each gantry position. Additional images (e.g., additional EPID images) are acquired while the gantry 102 remains fixed at 0° and the couch 106 rotates through a set of fixed couch angles.

The second step of the existing WL test includes analyzing the images described above to determine the LINAC geometric errors. This step can be further broken down into three sub-processes: (2a) using image processing techniques to find the radiation field and the marker 414 in each image (an example of an image 700 with the outer boundary 702 of the detected radiation field and the outer boundary 704 of the shadow of the marker 414 is shown in FIG. 7), (2b) finding the centroid of each contour (i.e., finding the centroid of the radiation field and the centroid of the marker 414), and (2c) computing the distance from the centroid of the marker 414 to the centroid of the field. FIG. 8 shows an example in which the geometric error is calculated using an image 800 (e.g., an EPID). In particular, the geometric error is calculated as the two-dimensional distance 810 from the centroid 806 of the radiation field 802 in the image 800 to the centroid 808 of the shadow 804 of the marker 414 in the image 800.

### 1.3 Multi-Target Single-Isocenter Treatment

Multi-target single-isocenter (SIMT) treatment is a radiation therapy technique in which multiple spatially distinct targets (e.g., brain metastases) are treated simultaneously using a single isocenter. Rather than aligning the treatment isocenter to each lesion individually, SIMT positions the isocenter at a fixed reference and uses multileaf collimator (MLC) shaping to conform the radiation beam to off-isocenter targets. This approach significantly improves treatment efficiency, reduces patient setup time, and minimizes intrafraction motion.

Ensuring geometric accuracy in SIMT is critical, as small misalignments can lead to significant dose delivery errors at off-isocenter locations due to rotational uncertainties and the increased sensitivity of beam targeting at greater distances from the isocenter.

### 1.4 Limitations of Current SIMT Quality Assurance Methods

Performing quality assurance (QA) for SIMT treatments is similar to the standard Winston-Lutz test, with the primary difference being that markers are positioned off-isocenter. A typical SIMT phantom is shown in FIGS. 9A-9C.

A key challenge in SIMT QA is the accurate positioning of multi-target phantoms, particularly in terms of angular orientation. Small angular errors in the phantom's positioning create large marker positioning errors, especially for markers distant from the LINAC's isocenter.

A further limitation is the requirement for square fields centered on the target. Since its introduction, SIMT QA has adhered to the “marker in the square field” model, an approach inherited from legacy WL testing. This constraint narrows the scope of field configurations being tested, potentially missing LINAC behaviors relevant to real-world SIMT treatments.

Improvements are needed to address these limitations of the SIMT QA method.

### SUMMARY

Aspects of the present invention may provide a system and method for quality assurance (QA) in single-isocenter, multi-target (SIMT) radiation therapy. Aspects of the invention may address one or more limitations of the existing SIMT QA method by enabling analysis of fields of any size or shape while incorporating six-degree-of-freedom phantom positioning and tracking.

One aspect of the present invention may provide a method including providing one or more radio-opaque markers at off-isocenter locations within a phantom. The method may include acquiring the actual position and orientation of the marker. The method may include defining an expected radiation field shape without requiring the marker to be centered within the field. The method may include applying a mathematical transformation to project the actual marker position into the beam's-eye view. The method may include acquiring a radiation transmission image. The method may include analyzing the measured field relative to the expected field using one or more analysis metrics.

Another aspect of the present invention may provide a method including providing a phantom containing one or more radio-opaque markers. The method may include acquiring MV or kV images of the phantom. The method may include processing the images to determine three-dimensional marker positions. The method may include calculating the rotational deviation of the phantom. The method may include adjusting the orientation or the analysis to account for the determined deviation.

Still another aspect of the present invention may provide a method of deriving the expected radiation field contour used as the reference in the quality assurance analysis from the dose distribution computed by the treatment planning system, rather than solely from the geometric aperture defined in the radiotherapy plan file. The dose-derived field contour may be extracted by applying an isodose threshold to the two-dimensional dose distribution calculated by the treatment planning system in the beam's-eye view plane at the depth of the marker, thereby producing a reference field boundary that incorporates beam modeling effects (e.g., penumbra, multileaf collimator (MLC) transmission, and/or tongue-and-groove). This dose-derived contour may be used in place of, or in combination with, the geometric aperture contour obtained from the radiotherapy plan file.

Yet another aspect of the present invention may provide a method in which the reference marker position used in the quality assurance analysis is derived directly from the treatment planning system, without requiring a separate physical measurement of phantom orientation. The reference marker position may be obtained from: (a) the centroid of the marker structure contour in the RT structure set generated during treatment planning or (b) the centroid of the marker volume as represented in the RT dose distribution computed by the treatment planning system. Because this method uses the planning system's own record of marker position rather than a corrected orientation measurement, this method may not remove phantom setup error from the analysis. The resulting geometric error therefore represents the combined accuracy of the LINAC delivery and the phantom setup, constituting an end-to-end system accuracy test. This may contrast with a method that isolates intrinsic LINAC geometric errors by explicitly removing phantom orientation errors through a rigid-body correction.

Still another aspect of the invention may provide a method for quality assurance in single-isocenter, multi-target (SIMT) radiation therapy. The method may include (a) using a measurement system to measure an actual marker position in a three-dimensional (3D) coordinate system of a linear accelerator (LINAC). The actual marker position may be a measured position of an off-isocenter radiation opaque marker of a phantom, and the off-isocenter radiation opaque marker may be offset from an isocenter of the LINAC in at least one spatial dimension. The method may include (b) using the actual marker position to compute a beam's eye view marker position. The beam's eye view marker position may be a projected position of the off-isocenter radiation opaque marker in an expected field contour of an expected radiation beam, and the expected radiation beam may be a radiation beam expected to be generated by the LINAC. The method may include (c) determining a centroid of the expected field contour. The method may include (d) using the LINAC to generate a radiation beam. The method may include (e) using an imaging device of the LINAC to acquire a two-dimensional (2D) radiation transmission image indicative of a radiation field of the generated radiation beam after passing through the off-isocenter radiation opaque marker of the phantom. The method may include (f) using the 2D radiation transmission image to determine (1) a measured field contour and (2) a measured marker contour. The measured field contour may be a boundary of the radiation field of the generated radiation beam in the 2D radiation transmission image, and the measured marker contour may be a boundary of a shadow of the off-isocenter radiation opaque marker in the radiation field of the generated radiation beam in the 2D radiation transmission image. The method may include (g) determining a centroid of the measured field contour and a centroid of the measured marker contour. The method may include (h) aligning the beam's eye view marker position with the centroid of the measured marker contour at a common marker reference point. The method may include (i) with the beam's eye view marker position aligned with the centroid of the measured marker contour at the common marker reference point, determining a geometric error. The geometric error may be a 2D distance between the centroid of the expected field contour and the centroid of the measured field contour.

In some aspects, the beam's eye view marker position may not coincide with a center of the expected field contour.

In some aspects, the method may further include deriving the expected field contour. In some aspects, the expected field contour may be derived based on one or both of: (1) geometric multileaf collimator (MLC) aperture and jaw settings specified in a radiotherapy plan file and projected into a beam's-eye view plane at a depth of the off-isocenter radiation opaque marker; and (2) an isodose contour extracted from a 2D dose distribution computed by a treatment planning system in the beam's-eye view plane at the depth of the off-isocenter radiation opaque marker.

In some aspects, using the measurement system to measure the actual marker position may include using the measurement system to measure the position and orientation of the phantom, and computing the beam's eye view marker position comprises applying a rigid-body transformation encoding three translational and three rotational degrees of freedom representing the measured position and orientation of the phantom to project the actual marker position into the beam's eye view of the expected field contour.

In some aspects, the method may further include performing a contour alignment comparison, computing a contour overlap metric, and/or performing a leaf-by-leaf multileaf collimator aperture comparison between the expected and measured field contours. In some aspects, the measurement system may include an optical tracking system, a kilovoltage imaging system, a megavoltage imaging system, a surface imaging device, and/or a laser distance measurement device. In some aspects, the imaging device may comprise an electronic portal imaging device (EPID).

In some aspects, steps (a) through (i) may be performed with respect to a first combination of gantry angle and couch angle, and the method may further include: repeating steps (b) through (f) for a plurality of different combinations of gantry angle and couch angle, and aggregating the geometric errors determined at each combination of gantry angle and couch angle to determine an overall geometric accuracy of the LINAC at the actual marker position.

In some aspects, the method may further include using the determined geometric error to perform at least one of: approving a patient treatment if the geometric error is below a clinical acceptance threshold; adjusting planning target volume margins based on the geometric error; and limiting treatment to off-isocenter target locations whose geometric error satisfies the clinical acceptance threshold.

Yet another aspect of the invention may provide a system for quality assurance in single-isocenter, multi-target (SIMT) radiation therapy. The system may include a phantom including an off-isocenter radiation opaque marker. The system may include a linear accelerator (LINAC) configured to generate a radiation beam. The LINAC may include an imaging device configured to acquire a two-dimensional (2D) radiation transmission image indicative of a radiation field of the generated radiation beam after passing through the off-isocenter radiation opaque marker of the phantom, and the off-isocenter radiation opaque marker may be offset from an isocenter of the LINAC in at least one spatial dimension. The system may include a measurement system configured to measure an actual marker position in a three-dimensional (3D) coordinate system of the LINAC. The actual marker position may be a measured position of the off-isocenter radiation opaque marker of the phantom, and the off-isocenter radiation opaque marker may be offset from an isocenter of the LINAC in at least one spatial dimension. The system may include processing circuitry. The processing circuitry may be configured to use the actual marker position to compute a beam's eye view marker position, the beam's eye view marker position may be a projected position of the off-isocenter radiation opaque marker in an expected field contour of an expected radiation beam, and the expected radiation beam may be a radiation beam expected to be generated by the LINAC. The processing circuitry may be configured to determine a centroid of the expected field contour. The processing circuitry may be configured to use the 2D radiation transmission image to determine (i) a measured field contour and (ii) a measured marker contour. The measured field contour may be a boundary of the radiation field of the generated radiation beam in the 2D radiation transmission image, and the measured marker contour may be a boundary of a shadow of the off-isocenter radiation opaque marker in the radiation field of the generated radiation beam in the 2D radiation transmission image. The processing circuitry may be configured to determine a centroid of the measured field contour and a centroid of the measured marker contour. The processing circuitry may be configured to align the beam's eye view marker position with the centroid of the measured marker contour at a common marker reference point. The processing circuitry may be configured to, with the beam's eye view marker position aligned with the centroid of the measured marker contour at the common marker reference point, determine a geometric error, and the geometric error may be a 2D distance between the centroid of the expected field contour and the centroid of the measured field contour.

Still another aspect of the invention may provide a method for quality assurance in single-isocenter, multi-target (SIMT) radiation therapy. The method may include (a) using a measurement system to acquire megavoltage, kilovoltage, and/or surface images of a phantom. The measurement system may include a kilovoltage imaging system, a megavoltage imaging system, and/or a surface imaging device, and the phantom may include one or more off-isocenter radiation opaque markers. The method may include (b) using the megavoltage, kilovoltage, and/or surface images to determine one or more three-dimensional (3D) positions of the one or more off-isocenter radiation opaque markers. The method may include (c) calculating a rotational deviation of the phantom, and calculating the rotational deviation of the phantom may include comparing the one or more determined 3D positions of the one or more off-isocenter radiation opaque markers with one or more expected 3D positions of the one or more off-isocenter radiation opaque markers. The method may include (d) using the calculated rotational deviation of the phantom to adjust an orientation of the phantom.

In some aspects, adjusting the orientation of the phantom may include physically adjusting the orientation of the phantom to reduce the rotational deviation of the phantom. In some aspects, adjusting the orientation of the phantom may include mathematically adjusting the orientation of the phantom in an analysis of delivered radiation fields. In some aspects, adjusting the orientation of the phantom may include computing one or more corrected 3D positions for each of the one or more off-isocenter radiation opaque markers by applying a rigid-body transformation encoding the calculated rotational deviation of the phantom to the one or more expected 3D positions of the one or more off-isocenter radiation opaque markers.

In some aspects, the 3D positions of the one or more off-isocenter radiation opaque markers may be determined by acquiring the megavoltage, kilovoltage, and/or surface images at two or more gantry angles and reconstructing 3D coordinates of the one or more off-isocenter radiation opaque markers through geometric back-projection.

In some aspects, the method may further include, for each off-isocenter radiation opaque marker of the one or more off-isocenter radiation opaque markers, (1) using the actual marker position to compute a beam's eye view marker position, the beam's eye view marker position may be a projected position of the off-isocenter radiation opaque marker in an expected field contour of an expected radiation beam associated with the off-isocenter radiation opaque marker, and the expected radiation beam may be a radiation beam expected to be generated by the LINAC. In some aspects, the method may further include, for each off-isocenter radiation opaque marker of the one or more off-isocenter radiation opaque markers, (2) determining a centroid of the expected field contour. In some aspects, the method may further include, for each off-isocenter radiation opaque marker of the one or more off-isocenter radiation opaque markers, (3) using the linear accelerator (LINAC) to generate a radiation beam. In some aspects, the method may further include, for each off-isocenter radiation opaque marker of the one or more off-isocenter radiation opaque markers, (4) using an imaging device of the LINAC to acquire a two-dimensional (2D) radiation transmission image indicative of a radiation field of the generated radiation beam after passing through the off-isocenter radiation opaque marker of the phantom. In some aspects, the method may further include, for each off-isocenter radiation opaque marker of the one or more off-isocenter radiation opaque markers, (5) using the 2D radiation transmission image to determine (i) a measured field contour and (ii) measured marker contour, the measured field contour may be a boundary of the radiation field of the generated radiation beam in the 2D radiation transmission image, and the measured marker contour may be a boundary of a shadow of the off-isocenter radiation opaque marker in the radiation field of the generated radiation beam in the 2D radiation transmission image. In some aspects, the method may further include, for each off-isocenter radiation opaque marker of the one or more off-isocenter radiation opaque markers, (6) determining a centroid of the measured field contour and a centroid of the measured marker contour. In some aspects, the method may further include, for each off-isocenter radiation opaque marker of the one or more off-isocenter radiation opaque markers, (7) aligning the beam's eye view marker position with the centroid of the measured marker contour at a common marker reference point. In some aspects, the method may further include, for each off-isocenter radiation opaque marker of the one or more off-isocenter radiation opaque markers, (8) with the beam's eye view marker position aligned with the centroid of the measured marker contour at the common marker reference point, determining a geometric error, and the geometric error is a 2D distance between the centroid of the expected field contour and the centroid of the measured field contour.

In some aspects, for each off-isocenter radiation opaque marker of the one or more off-isocenter radiation opaque markers, computing the beam's eye view marker position may include applying a mathematical transformation to project the actual marker position into a beam's eye view of the expected field contour.

Yet another aspect of the invention may provide a system for quality assurance in single-isocenter, multi-target (SIMT) radiation therapy. The system may include a phantom including one or more off-isocenter radiation opaque markers. The system may include a measurement system configured to acquire megavoltage, kilovoltage, and/or surface images of a phantom, and the measurement system may include a kilovoltage imaging system, a megavoltage imaging system, and/or a surface imaging device. The system may include processing circuitry configured to: (1) use the megavoltage, kilovoltage, and/or surface images to determine one or more three-dimensional (3D) positions of the one or more off-isocenter radiation opaque markers; and (2) calculate a rotational deviation of the phantom. Calculating the rotational deviation of the phantom may include comparing the one or more determined 3D positions of the one or more off-isocenter radiation opaque markers with one or more expected 3D positions of the one or more off-isocenter radiation opaque markers.

Still another aspect of the invention may provide a method for generating an expected field contour for use in quality assurance of single-isocenter, multi-target (SIMT) radiation therapy treatments. The method may include (a) obtaining, from a treatment planning system (TPS), a two-dimensional (2D) dose distribution computed in a beam's-eye view plane at a depth of an off-isocenter radiation opaque marker of a phantom. The off-isocenter radiation opaque marker may be offset from an isocenter of a linear accelerator (LINAC) in at least one spatial dimension. The method may include (b) extracting an isodose contour by applying an isodose threshold to the 2D dose distribution. The method may include (c) using the extracted isodose contour as the expected field contour against which a 2D radiation transmission image acquired by an imaging device of the LINAC is compared to determine a geometric accuracy of radiation field delivery at a position of the off-isocenter radiation opaque marker.

In some aspects, the isodose threshold may be a 50% isodose level normalized to one of: a prescription dose, a field maximum dose, and a user-specified reference dose. In some aspects, the method may further include comparing the isodose contour to a geometric aperture contour derived from multileaf collimator (MLC) leaf positions and jaw settings specified in a radiotherapy plan file, the geometric aperture contour may be used to assess MLC positioning accuracy, and the isodose contour may be used to assess dosimetric field accuracy at the position of the off-isocenter radiation opaque marker.

In some aspects, the 2D dose distribution may be (i) extracted from a digitally-reconstructed radiograph created by the TPS, (ii) extracted from a three-dimensional (3D) distribution using ray tracing techniques along the source to imaging panel directions, or (iii) extracted from one or more predicted portal dose maps derived from the TPS.

Still another aspect of the invention may provide a method for end-to-end quality assurance in single-isocenter, multi-target (SIMT) radiation therapy. The method may include (a) obtaining a reference marker position from a treatment planning system (TPS). The reference marker position may be a derived position of an off-isocenter radiation opaque marker of a phantom, and the off-isocenter radiation opaque marker may be offset from an isocenter of a linear accelerator (LINAC) in at least one spatial dimension. The reference marker position may be derived from one of: (i) a centroid of a marker structure contour defined in a radiotherapy (RT) structure set generated during treatment planning and (ii) a centroid of a volume of the off-isocenter radiation opaque marker as represented in an RT dose distribution computed by the TPS. The method may include (b) projecting the reference marker position into a beam's-eye view corresponding to a planned gantry angle and couch angle of the LINAC, without applying a correction for physical positioning errors of the phantom. The method may include (c) using the LINAC to generate a radiation beam. The method may include (d) using an imaging device of the LINAC to acquire a two-dimensional (2D) radiation transmission image indicative of a radiation field of the generated radiation beam after passing through the off-isocenter radiation opaque marker of the phantom. The method may include (e) using the 2D radiation transmission image to determine (1) a measured field contour and (2) a measured marker contour. The measured field contour may be a boundary of the radiation field of the generated radiation beam in the 2D radiation transmission image, and the measured marker contour may be a boundary of a shadow of the off-isocenter radiation opaque marker in the radiation field of the generated radiation beam in the 2D radiation transmission image. The method may include (f) determining a centroid of the measured field contour and a centroid of the measured marker contour. The method may include (g) aligning the reference marker position with the centroid of the measured marker contour at a common marker reference point. The method may include (h) with the reference marker position aligned with the centroid of the measured marker contour at the common marker reference point, determining a geometric error. The geometric error may be a 2D distance between the centroid of an expected field contour of an expected radiation beam and the centroid of the measured field contour, and the expected radiation beam may be a radiation beam expected to be generated by the LINAC.

In some aspects, the reference marker position may be derived from the centroid of the marker structure contour in the RT structure set, and the RT structure set may have been generated from a planning computed tomography image of the phantom acquired prior to the quality assurance session.

In some aspects, the reference marker position may be derived from the centroid of the volume of the off-isocenter radiation opaque marker in the RT dose distribution computed by the TPS, and the RT dose distribution may have been computed using a beam geometry and multileaf collimator (MLC) aperture sequences that are the same as a beam geometry and MLC aperture sequences used for the generated radiation field.

In some aspects, steps (a) through (h) may be performed with respect to a first combination of gantry angle and couch angle, and the method may further include repeating steps (a) through (h) for a plurality of different combinations of gantry angle and couch angle. In some aspects, the method may further include determining geometric errors using an orientation-corrected method for quality assurance in SIMT radiation therapy on a 2D radiation transmission image dataset that is the same as a 2D radiation transmission image dataset used in the method for end-to-end quality assurance in SIMT radiation therapy; comparing the geometric errors produced by the method for end-to-end quality assurance in SIMT radiation therapy with the geometric errors produced by the orientation-corrected method; and attributing differences between the geometric errors to phantom setup error, thereby decomposing the total end-to-end system error into its LINAC geometric error component and its phantom setup error component.

Yet another aspect of the invention may provide a system for end-to-end quality assurance in single-isocenter, multi-target (SIMT) radiation therapy. The system may include a phantom, a linear accelerator (LINAC), and processing circuitry, The phantom may include an off-isocenter radiation opaque marker. The LINAC may be configured to generate a radiation beam. The LINAC may include an imaging device configured to acquire a two-dimensional (2D) radiation transmission image indicative of a radiation field of the generated radiation beam after passing through the off-isocenter radiation opaque marker of the phantom, and the off-isocenter radiation opaque marker may be offset from an isocenter of the LINAC in at least one spatial dimension. The processing circuitry may be configured to obtain a reference marker position from a treatment planning system (TPS). The reference marker position may be a derived position of the off-isocenter radiation opaque marker of the phantom. The reference marker position may be derived from one of: (i) a centroid of a marker structure contour defined in a radiotherapy (RT) structure set generated during treatment planning and (ii) a centroid of a volume of the off-isocenter radiation opaque marker as represented in an RT dose distribution computed by the TPS. The processing circuitry may be configured to project the reference marker position into a beam's-eye view corresponding to a planned gantry angle and couch angle of the LINAC, without applying a correction for physical positioning errors of the phantom. The processing circuitry may be configured to use the 2D radiation transmission image to determine (1) a measured field contour and (2) a measured marker contour. The measured field contour may be a boundary of the radiation field of the generated radiation beam in the 2D radiation transmission image, and the measured marker contour may be a boundary of a shadow of the off-isocenter radiation opaque marker in the radiation field of the generated radiation beam in the 2D radiation transmission image. The processing circuitry may be configured to determine a centroid of the measured field contour and a centroid of the measured marker contour. The processing circuitry may be configured to align the reference marker position with the centroid of the measured marker contour at a common marker reference point. The processing circuitry may be configured to, with the reference marker position aligned with the centroid of the measured marker contour at the common marker reference point, determine a geometric error that is a 2D distance between the centroid of an expected field contour of an expected radiation beam and the centroid of the measured field contour. The expected radiation beam may be a radiation beam expected to be generated by the LINAC.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 illustrates a medical linear accelerator (LINAC).

FIG. 2 illustrates rotation of a gantry of the LINAC.

FIG. 3 illustrates rotation of a couch of the LINAC.

FIG. 4 illustrates a radiation beam and imaging panel of the LINAC.

FIG. 5 illustrates a marker assembly.

FIG. 6 illustrates an unprocessed radiation transmission image from a standard Winston-Lutz acquisition.

FIG. 7 illustrates a processed radiation transmission image.

FIG. 8 illustrates a conventional Winston-Lutz geometric error measurement.

FIGS. 9A, 9B, and 9C illustrate perspective, top, and side views of a representative single-isocenter, multi-target (SIMT) quality assurance (QA) phantom according to some aspects.

FIG. 9D illustrates an SIMT QA phantom assembly and a measurement system according to some aspects.

FIG. 10 is a top view illustrating the effect of phantom angular misalignment on marker position in the SIMT QA phantom according to some aspects.

FIG. 11 illustrates a beam's-eye-view of expected field contour with nominal and beam's eye view marker positions according to some aspects.

FIG. 12 illustrates detected field and marker contours in a transmission image for an off-isocenter field configuration according to some aspects.

FIG. 13 illustrates expected and measured field contours aligned at the common marker reference point according to some aspects.

FIG. 14 illustrates a controller of a LINAC according to some aspects.

FIG. 15 illustrates an apparatus according to some aspects.

FIG. 16 is a flowchart illustrating a process for quality assurance in single-isocenter, multi-target (SIMT) radiation therapy according to some aspects.

FIG. 17 is a flowchart illustrating a process for quality assurance in single-isocenter, multi-target (SIMT) radiation therapy according to some aspects.

FIG. 18 is a flowchart illustrating a process for generating an expected field contour for use in quality assurance of single-isocenter, multi-target (SIMT) radiation therapy treatments according to some aspects.

FIG. 19 is a flowchart illustrating a process for end-to-end quality assurance in single-isocenter, multi-target (SIMT) radiation therapy according to some aspects.

### DETAILED DESCRIPTION OF PREFERRED EMBODIMENTS

### 2.1 LINAC Architecture

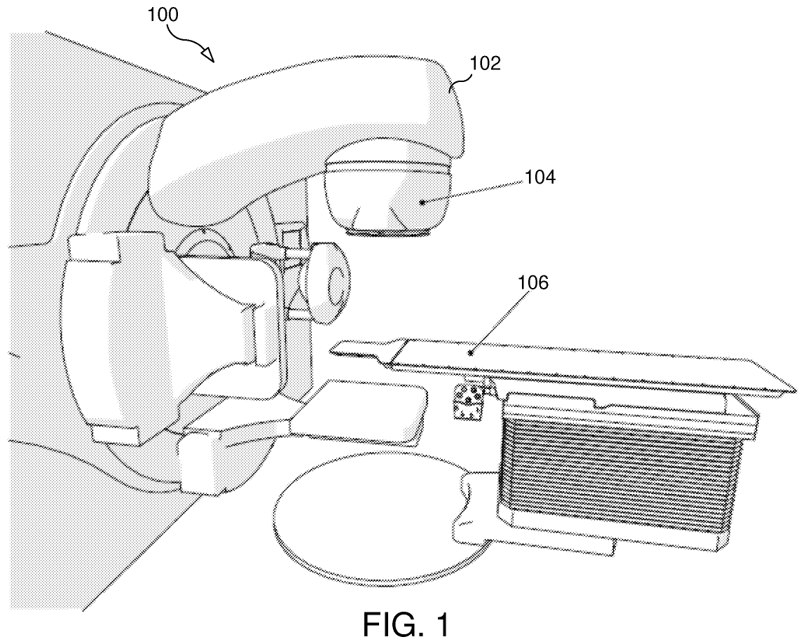

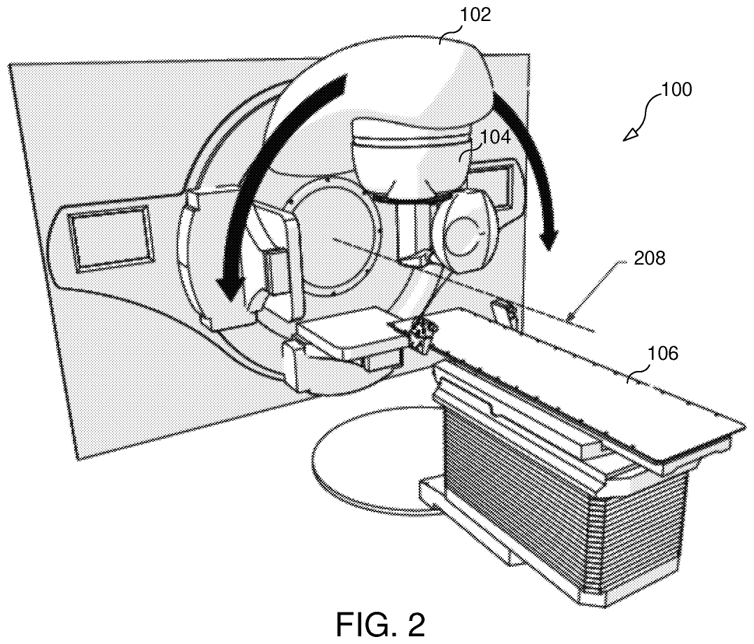

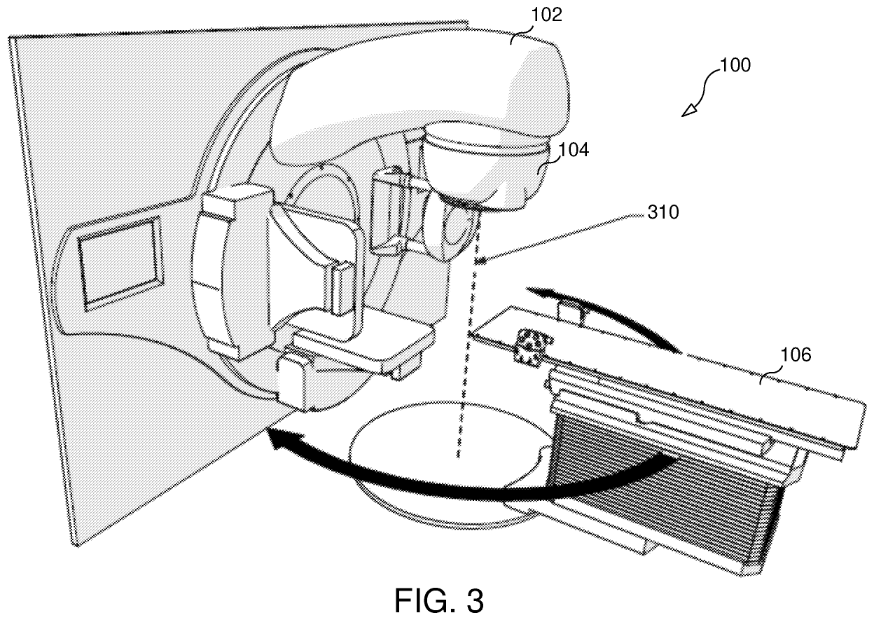

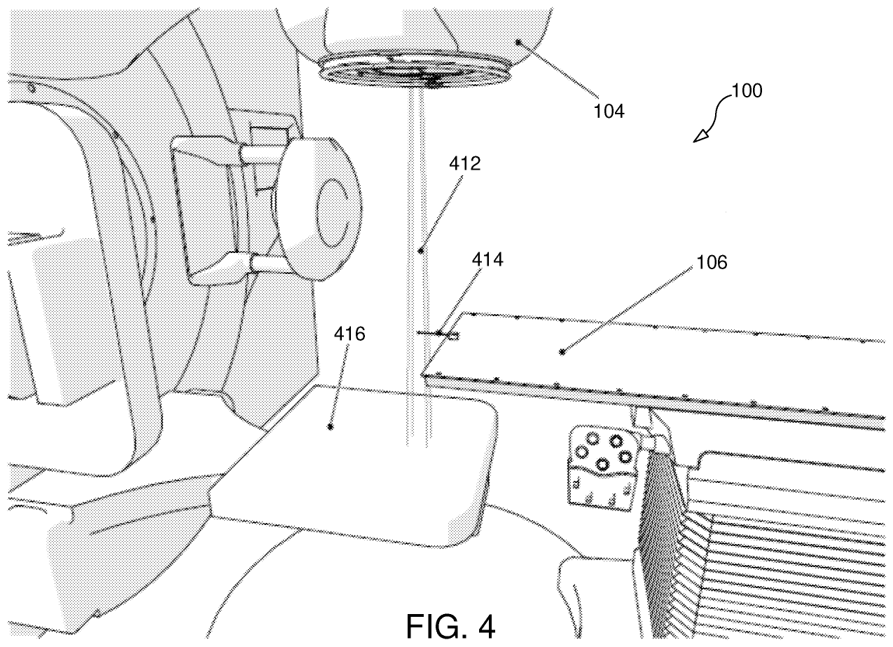

As shown in FIGS. 1-3, a medical linear accelerator (LINAC) 100 may include a gantry 102 and a treatment table or couch 106. In some aspects, as shown in FIGS. 1-3, the gantry 102 may include a collimator head 104. In some aspects, as shown in FIG. 4, the collimator head 104 may generate and shape a therapeutic radiation beam 412. In some aspects, the couch 106 may be configured to support a patient, who may be positioned on the couch 106. In some aspects, as shown in FIG. 2, the gantry 104 may be configured to rotate through a full 360° about a gantry axis of rotation 208, which may enable the radiation beam to be directed toward the patient from any angle within the plane of rotation of the gantry 102. In some aspects, as shown in FIG. 3, the treatment couch 106 may be configured to rotate about a couch axis of rotation 310 (e.g., a vertical axis), which may allow the patient's orientation to be adjusted relative to the gantry plane. In some aspects, an isocenter of the LINAC 100 may be the point in space that minimizes a maximum geometric error across all gantry and couch angle combinations.

### 2.2 Standard Winston-Lutz Test

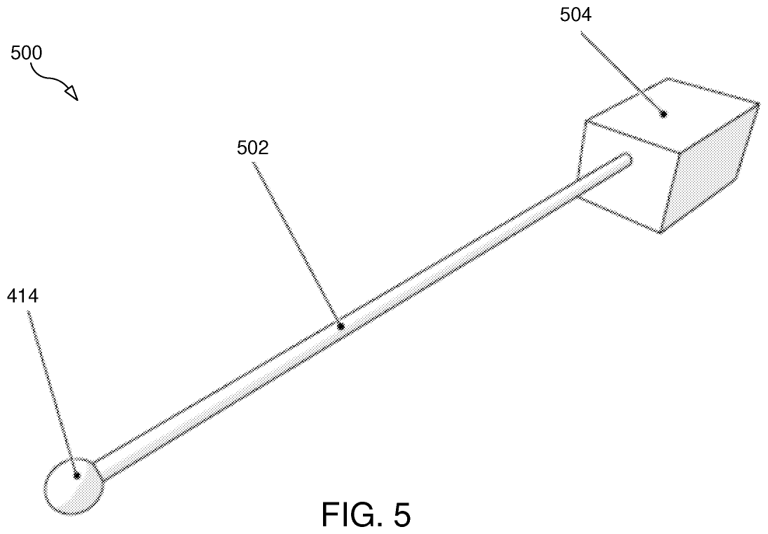

As shown in FIG. 4, during the existing Winston-Lutz test, the LINAC 100 may deliver a collimated radiation beam 412 through a radiation opaque marker 414 positioned at the isocenter of the LINAC 100. The imaging device 416 acquires a two-dimensional transmission image of the radiation beam 412 as it passes through the marker 414. A detailed view of a marker assembly 500 including the radiation opaque marker 414 is shown in FIG. 5. In FIG. 5, a high density marker 414 is connected to a low density support rod 502 that then connects to a base 504. The marker 414 may be spherical. The marker 414 may be fabricated from a high-density material (e.g., tungsten) that substantially attenuates the therapeutic x-ray beam and produces a detectable shadow in the image. The low density (e.g., plastic) support rod 502 may be a thin cylindrical rod that mechanically supports the marker 414 while minimally attenuating the radiation beam 412. The base 504 is set on a top of the treatment table or couch 106 such that the treatment table or couch 106 supports the marker assembly 500, and the marker 414 may be positioned at the isocenter of the LINAC 100.

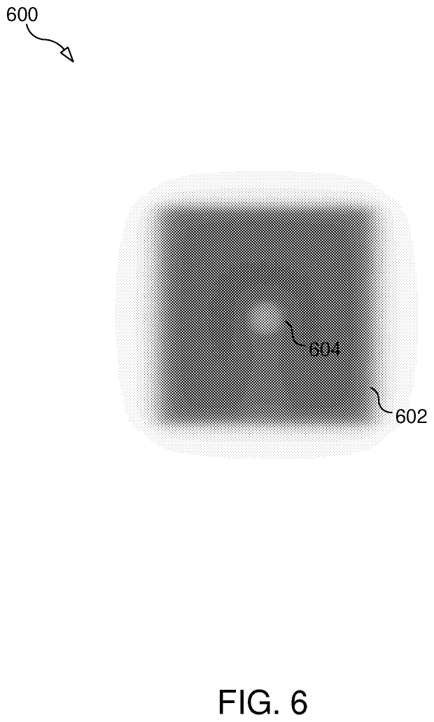

FIG. 6 shows a typical image 600 (e.g., an EPID image) through the marker 414. In FIG. 6, the dark square region 602 is created by a square radiation field of the radiation beam 412 exposing the imaging device 410, and the lighter inner circular shape 604 is created by the shadow of the radiation opaque marker 414 that is located within the radiation field.

Measuring the geometric errors of the LINAC 100 involves rotating the gantry 102 through a set of fixed gantry angles while the couch 106 remains at 0° and acquiring an image (e.g., an EPID image) for each gantry position. Additional images (e.g., additional EPID images) are acquired while the gantry 102 remains fixed at 0° and the couch 106 rotates through a set of fixed couch angles.

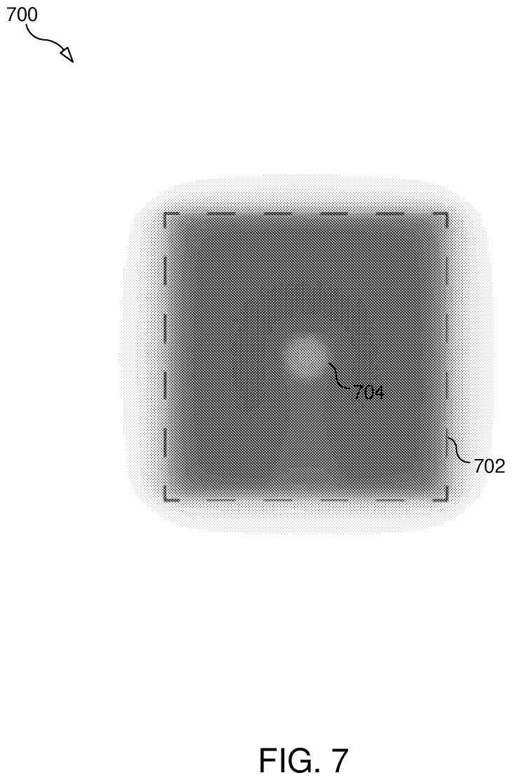

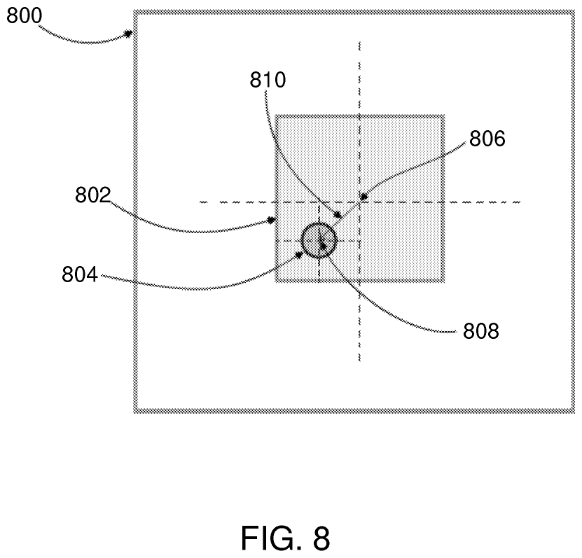

The second step of the existing WL test includes analyzing the images described above to determine the geometric errors of the LINAC 100. This step can be further broken down into sub-processes: (2a) using image processing techniques to find the outer boundary 702 of the radiation field and the outer boundary 704 of the shadow of the marker 414 in each image (an example of an image 700 with the outer boundary 702 of the detected radiation field and the outer boundary 704 of the shadow of the marker 414 is shown in FIG. 7), (2b) finding the centroid of field boundary and marker contours (i.e., finding the centroid of the outer boundary 702 of the radiation field and the centroid of the outer boundary 704 of the shadow of the marker 414), and (2c) computing geometric error as the distance from the centroid of the marker 414 to the centroid of the radiation field. FIG. 8 shows an example of the conventional WL test in which the geometric error is calculated using an image 800 (e.g., an EPID). In particular, the geometric error is calculated as the two-dimensional distance 810 from the centroid 806 of the radiation field 802 in the image 800 to the centroid 808 of the shadow 804 of the marker 414 in the image 800.

### 2.3 Single-Isocenter, Multi-Target (SIMT) Phantom

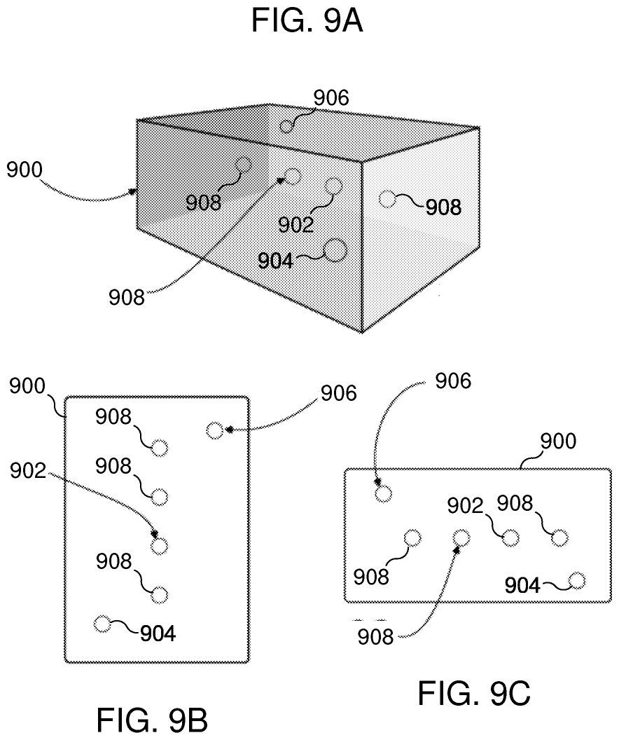

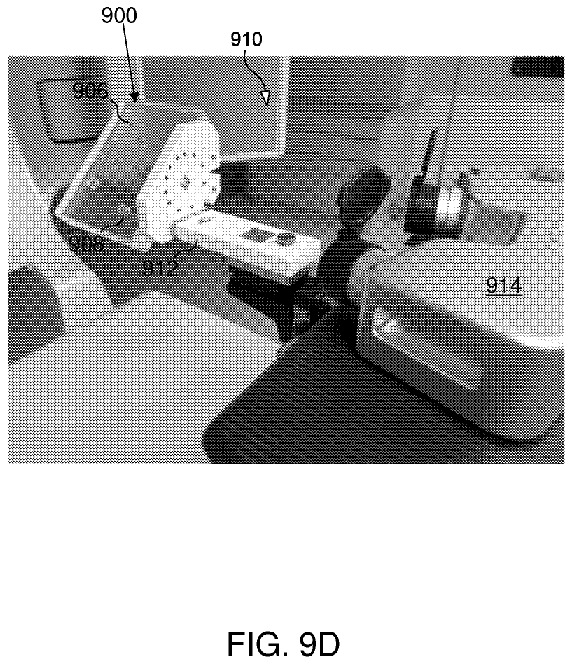

FIGS. 9A-9C illustrate perspective, side, and top views, respectively, of a single-isocenter, multi-target (SIMT) quality assurance (QA) phantom 900 according to some aspects. In some aspects, as shown in FIGS. 9A-9C, the phantom 900 may include one or more radiation opaque markers 902, 904, 906, 908. In some aspects, the one or more markers 902, 904, 906, 908 of the phantom 900 may be fabricated from a high-density material (e.g., tungsten) that substantially attenuates the radiation beam 414 generated by the collimator head 104 of the LINAC 100. In some aspects, the one or more markers 902, 904, 906, 908 of the phantom 900 may be embedded in a low density material (e.g., plastic, such as, acrylic) that minimally attenuates the radiation beam 412.

In some aspects, the one or more radiation opaque markers 902, 904, 906, and 908 may include an isocenter-aligned marker 902 that is a radiation opaque marker intended to be positioned at (or near) the isocenter of the LINAC 100. In some aspects, the one or more radiation opaque markers 902, 904, 906, and 908 may additionally or alternatively include one or more off-isocenter markers 904, 906, and 908, which may each be offset from the isocenter in at least one spatial dimension. In some aspects, the one or more off-isocenter markers 904, 906, and 908 may include a first off-isocenter marker 904. In some aspects, the first off-isocenter marker 904 may be offset from the isocenter in all three spatial dimensions. In some aspects, the one or more off-isocenter markers 904, 906, and 908 may additionally or alternatively include a second off-isocenter marker 906. In some aspects, the second off-isocenter marker 906 may be offset from the isocenter in all three spatial dimensions. In some aspects including first and second off-isocenter markers 904 and 906, the first off-isocenter marker 904 may be in different portions of the phantom 900. For example, in some aspects including first and second off-isocenter markers 904 and 906, as shown in FIGS. 9A-9C, the first off-isocenter marker 904 may be in an anterior portion of the phantom 900, and the second off-isocenter marker 906 may be in a posterior portion of the phantom 900. In some aspects, the one or more off-isocenter markers 904, 906, and 908 may additionally or alternatively include one or more third off-isocenter markers 908, which may each be offset from the isocenter in one or two spatial dimensions. In some aspects, the one or more third off-isocenter markers 908 may all be offset from the isocenter in the same spatial dimension. In some aspects, the third off-isocenter markers 908 may be in a row with the isocenter-aligned marker 902. In some aspects, the radiation opaque markers 902 and 908 in the row may be spaced apart from one another (e.g., by 30 mm).

In some aspects, as shown in FIGS. 9A-9C, some or all of the radiation opaque markers 902, 904, 906, 908 may be spherical. However, this is not required, and, in some alternative aspects, different shapes (e.g., rectangular prism, diamond, oval, or cylinder) may be used for one or more (or all) of the radiation opaque markers 902, 904, 906, 908. For example, in some alternative aspects, the markers 904 and 906 may have a different shape than the markers 902 and 908 (e.g., to make the markers 904 and 906 easier to distinguish from the markers 902 and 908).

In some aspects, as shown in FIG. 9D, the SIMT QA phantom 900 may be part of a SIMT QA phantom assembly 910. In some aspects, the SIMT QA phantom assembly 910 may include a support 912 for the phantom 900. In some aspects, a measurement system 914 may be configured to acquire images of the phantom 900 three-dimensional (3D) positions of the one or more radiation opaque markers 902, 904, 906, 908 may be determined. In some aspects, as shown in FIG. 9D, the measurement system 914 may include an optical tracking system. In some aspects, the measurement system 914 may additionally or alternatively include a kilovoltage imaging system, a megavoltage imaging system, a surface imaging device, and/or a laser distance measurement device.

### 2.4 Phantom Positioning and Orientation Measurement

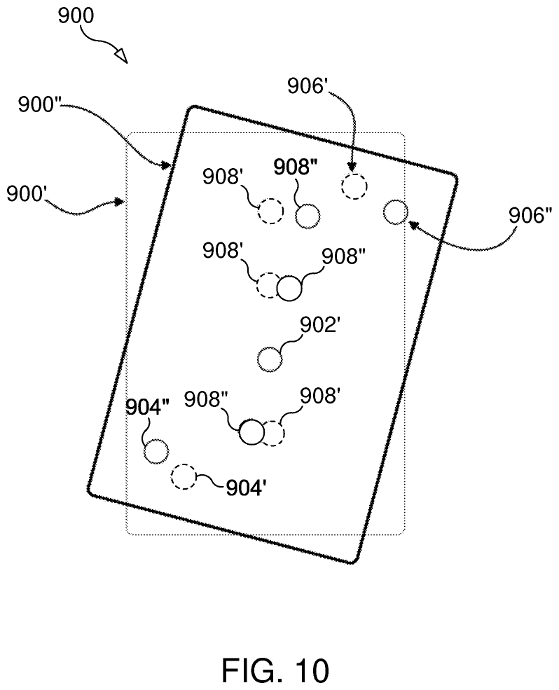

FIG. 10 illustrates the SIMT QA phantom 900 in both a perfectly aligned orientation 900′ and in a rotationally misaligned orientation 900″. In some aspects, the perfectly aligned orientation 900′ may be the ideal orientation of the SIMT QA phantom 900, with no rotational deviation from its intended setup position. In some aspects, in the perfectly aligned orientation 900', the SIMT QA phantom 900 may be perfectly aligned to the LINAC coordinate system. In some aspects, as shown in FIG. 10, in the perfectly aligned orientation 900′, the one or more radiation opaque markers 902, 904, 906, 908 of the SIMT QA phantom 900 may be in expected positions 902′, 904′, 906′, and 908′, respectively. In some aspects, no positional error is present under the ideal condition in which the SIMT QA phantom 900 is in the perfectly aligned orientation 900′.

In some aspects, in the rotationally misaligned orientation 900″, the SIMT QA phantom 900 may be in an angularly misaligned orientation relative to the LINAC coordinate system. In some aspects, as shown in FIG. 10, in the rotationally misaligned orientation 900″, the isocenter-aligned marker 902 may still be in the expected position 902′. In some aspects, as shown in FIG. 10, in the rotationally misaligned orientation 900″, the one or more off-isocenter markers 904, 906, and 908 of the SIMT QA phantom 900 may have shifted from the expected positions 904′, 906′, and 908′, respectively, and may instead be in displaced positions 904″, 906″, and 908″, respectively. In some aspects, the magnitude of the deviation between the expected and displaced positions (i.e., the positional error) may increase with distance from the axis of rotation.

In some aspects, an SIMT QA process may include measuring the position and orientation of the SIMT QA phantom 900 prior to or concurrent with radiation field delivery. In some aspects, suitable measurement techniques may include, for example and without limitation, optical tracking systems, LINAC-based kilovoltage (kV) or megavoltage (MV) imaging, surface imaging, and/or laser distance measurement devices.

In some aspects, an SIMT QA process may include, after measuring the position and orientation of the SIMT QA phantom 900, computing the three-dimensional position of one or more of the one or more radiation opaque markers 902, 904, 906, 908 (e.g., one or more of the one or more off-isocenter markers 904, 906, and 908) by applying the measured rigid-body transformation to the known nominal marker coordinates.

### 2.5 Beam's-Eye-View Projection

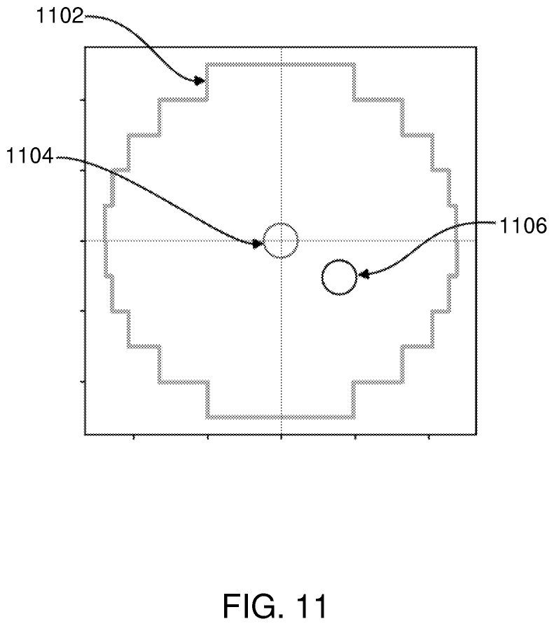

FIG. 11 shows an expected field contour 1102 according to some aspects. In some aspects, the expected field contour 1102 may be the reference two-dimensional field boundary in the beam's-eye view against which the measured delivery is compared. In some aspects, the expected field contour 1102 may be derived from one or both of: (i) a radiotherapy (RT) plan file aperture contour and (ii) a dose-derived field contour.

In some RT plan file aperture contour aspects, the RT plan file aperture contour may include multileaf collimator (MLC) leaf position sequence and jaw settings that define the geometric aperture of the planned field, and the expected field contour 1102 may be derived from the MLC leaf position sequence and jaw settings. In some RT plan file aperture contour aspects, the aperture contour may be projected into the beam's eye view at a plane/depth of an off-isocenter radiation opaque marker of the one or more off-isocenter markers 904, 906, or 908 of the phantom 900. In some RT plan file aperture contour aspects, standard divergent projection geometry may be used to project the aperture contour into the beam's eye view at the plane of the off-isocenter radiation opaque marker. In some RT plan file aperture contour aspects, the RT plan file aperture contour may represent an intended mechanical aperture of the radiation field, and the RT plan file aperture contour may be independent of beam modeling.

In some dose-derived field contour aspects, the expected field contour 1102 may be extracted from the two-dimensional (2D) dose distribution computed by the treatment planning system (TPS) in the beam's-eye view plane at the depth of the off-isocenter radiation opaque marker 904, 906, or 908 of the phantom 900. In some aspects, the 2D dose distribution may be extracted from a digitally-reconstructed radiograph (DRR) computed at the imaging device location and projected back to isocenter. In some aspects, the 2D dose distribution may be extracted from one or more predicted portal dose maps derived from the TPS. In some dose-derived field contour aspects, the TPS dose distribution may incorporate beam modeling effects such as, for example and without limitation, photon beam penumbra, MLC leaf transmission, MLC tongue-and-groove effects, and/or head scatter. In some dose-derived field contour aspects, an isodose contour may be extracted from this dose plane by applying a threshold (e.g., a 30-70% or 40-60% isodose level, such as a 50% isodose level, normalized to the prescription dose or the field maximum), thereby producing a reference field boundary that reflects the actual expected dose profile rather than the geometric aperture alone. In some aspects, the dose-derived field contour may be a more physically accurate reference for comparison with the EPID-measured transmission image, because the EPID may also respond to transmitted and scattered radiation in a manner that more closely follows the dose distribution than the geometric aperture edge.

In some aspects, the two sources (i.e., the geometric aperture contour and the dose-derived contour) may be used independently or in combination to derive the expected field contour 1102. In some aspects that use a combination of the two sources to derive the expected field contour 1102, the geometric aperture contour may serve as a check on the MLC positioning accuracy, and the dose-derived contour may serve as a check on the overall dosimetric field accuracy. In some aspects, the choice of contour source, the isodose threshold applied, and the depth at which the dose plane is extracted may be configurable parameters of the method.

FIG. 11 shows a nominal marker position 1104 according to some aspects. In some aspects, the nominal marker position 1104 may be the position of the off-isocenter radiation opaque marker 904, 906, or 908 of the phantom 900 in the beam's-eye view under the assumption of perfect placement (e.g., orientation) of the phantom 900. In the convention WL test, the nominal marker position 1104 coincides with the center of the radiation field.

FIG. 11 also shows a beam's eye view marker position 1106 according to some aspects. In some aspects, the beam's eye view may be a 2D plane perpendicular to a central axis of a radiation beam 412 generated by the LINAC 100. In some aspects, the beam's eye view marker position 1106 may be the position of the off-isocenter radiation opaque marker 904, 906, or 908 of the phantom 900 in the beam's-eye view computed by applying a measured rigid-body transformation, which may account for actual positioning errors. The beam's eye view marker position 1106 need not coincide with the center of the radiation field. In some aspects, the beam's eye view marker position 1106 and the expected field contour 1102 may together define the reference geometry and may eliminate the requirement that the marker be centered within the radiation field.

### 2.6 TPS-Derived Marker Reference Position—End-to-End Test Workflow

In some orientation-corrected aspects, an SIMT QA process may isolate intrinsic LINAC geometric errors by applying a rigid-body correction that removes phantom orientation error, yielding a measurement of the LINAC's geometric accuracy alone. In some alternative aspects, the reference marker position may be derived directly from the treatment planning system (TPS) rather than from a physical orientation measurement. In some TPS-derived marker reference position aspects, the phantom setup error may be intentionally retained in the analysis and thereby constitutes an end-to-end system accuracy test, which may measure the combined geometric accuracy of the LINAC delivery and the phantom setup together.

In some TPS-derived marker reference position aspects, the reference marker position in the beam's-eye view may be determined from one of two sources within the treatment planning system: (1) a radiotherapy (RT) structure set centroid and (2) an RT dose distribution centroid. In some RT structure set centroid aspects, the reference marker position may be taken as the centroid of the marker structure contour as delineated in the RT structure set generated during treatment planning. In some RT structure set centroid aspects, the marker structure contour may be drawn on the planning CT image of the phantom, and the marker structure contour may represent the planned geometric location of the marker relative to the treatment isocenter. In some RT dose distribution centroid aspects, the reference marker position may be taken as the centroid of the marker volume as represented in the three-dimensional RT dose distribution computed by the TPS. In some RT dose distribution centroid aspects, the RT dose distribution centroid may reflect both the planned marker location and any dose modeling effects applied by the TPS.

In some TPS-derived marker reference position aspects, the reference marker position may be projected into the beam's-eye view using the planned gantry and couch geometry (e.g., without applying any correction for physical phantom positioning errors). In some aspects, the geometric error may then be computed as the distance between the centroid of the measured field (e.g., from the EPID image) and the centroid of the expected field, after aligning the TPS-derived reference marker position with the measured marker centroid at a common reference point.

In some TPS-derived marker reference position aspects, because no orientation correction is applied, any angular or translational setup error in the physical placement of the phantom may contribute directly to the measured geometric error. In some TPS-derived marker reference position aspects, the measured geometric error may therefore reflect the total end-to-end system accuracy: the quadrature combination of LINAC geometric error and phantom setup error. This may be in contrast to orientation-corrected aspects that remove phantom setup error and measure the LINAC geometric error in isolation.

In some aspects, the-corrected aspects and the TPS-derived marker reference position aspects may be complimentary and may be used in combination (e.g., applied to the same dataset). In some aspects, the orientation-corrected process may report pure LINAC geometric error and may used to evaluate machine performance, and the TPS-reference process may report end-to-end system error and may be used to evaluate the complete treatment delivery chain (e.g., including patient setup reproducibility).

### 2.7 Image Acquisition and Contour Detection

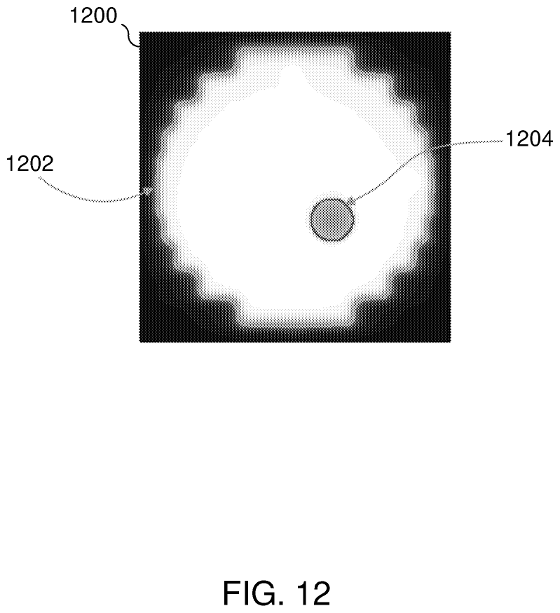

FIG. 12 is a radiation transmission image 1200 (e.g., an EPID radiation transmission image) showing detected field and marker contours 1202 and 1204 for an off-isocenter field configuration according to some aspects. In some aspects, an image such as the image 1200 shown in FIG. 12 may be acquired for each delivered field. In some aspects, as shown in FIG. 12, the image 1200 may be processed to identify a measured field contour 1202 and a measured marker contour 1204. In some aspects, the measured field contour 1202 may be the detected boundary of the delivered radiation field, and the measured field centroid may be computed from the enclosed area of the measured field contour 1202. In some aspects, the measured marker contour 1204 may be the detected boundary of the marker shadow, and the marker shadow centroid may be computed from the measured marker contour 1204. In some aspects, as shown in FIG. 12, the marker may appear at an arbitrary location within the field contour.

### 2.8 Geometric Error Quantification

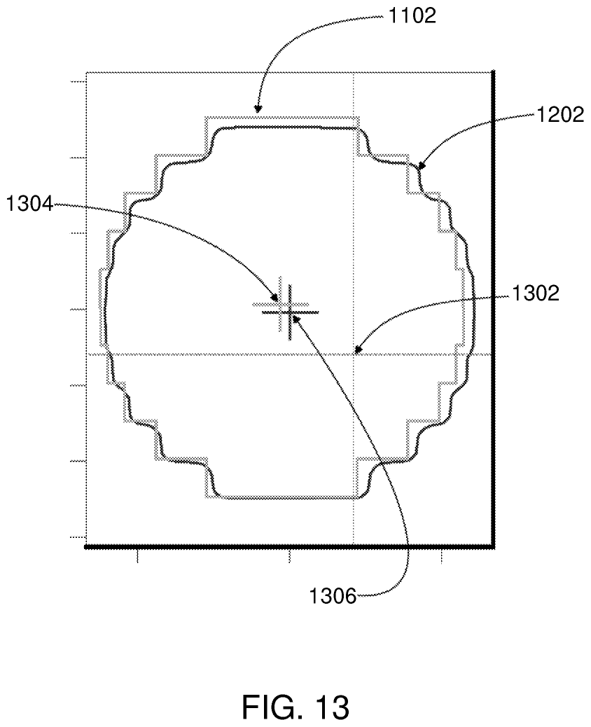

FIG. 13 illustrates a geometric error analysis according to some aspects. In some aspects, the corrected expected marker position and the measured marker centroid may be aligned at a common marker reference point 1302. In some aspects, the common marker reference point 1302 may establish a shared coordinate origin for comparing expected and measured field positions. In some aspects, as shown in FIG. 13, the expected field contour 1102 may be overlaid on the measured field contour 1202. In some aspects, the expected field contour 1102 may be the planned field boundary from the TPS positioned relative to the common reference point 1302. In some aspects, the measured field contour 1202 may be the field boundary as detected from the EPID image positioned relative to the common reference point 1302. In some aspects, an expected field centroid 1304 may be the geometric centroid of the expected field contour 1102. In some aspects, the expected field centroid 1304 may represent an intended delivery position. In some aspects, a measured field centroid 1306 may be the geometric centroid of the measured field contour 1202. In some aspects, the measured field centroid 1306 may represents the actual delivery position. In some aspects, the geometric error may be determined as the two-dimensional distance between the expected field centroid 1304 and the measured field centroid 1306, which may be corrected for phantom positioning error. In some aspects, additional metrics (e.g., contour alignment, overlap comparison, and/or leaf-by-leaf MLC aperture comparison) may be determined.

### 2.9 Clinical Decision Support

In some aspects, the measured geometric errors may be used to, for example and without limitation, (a) approve treatment delivery if the geometric error at each target falls below a predefined clinical acceptance threshold, (b) adjust planning target volume margins based on the determined geometric accuracy, and/or (c) limit treatment to only those off-isocenter targets whose measured geometric error satisfies the clinical acceptance threshold.

### 2.10 Phantom Orientation Correction via LINAC Imaging

In some aspects, three-dimensional marker positions of the one or more radiation opaque markers 902, 904, 906, 908 of the SIMT QA phantom 900 may be determined directly from megavolt (MV) or kilovolt (kV) images or surface images acquired by the LINAC's imaging device 416 and/or measurement system 914. In some aspects, by acquiring images at two or more gantry angles, the three-dimensional marker coordinates may be reconstructed through, for example, geometric back-projection. In some aspects, the rotational deviation of the phantom 900 may then be calculated by comparing the measured coordinates to the known nominal coordinates. In some aspects, the phantom orientation may then be adjusted (e.g., either physically or mathematically within the analysis) to minimize systematic positioning errors.

### 2.11 LINAC Controller

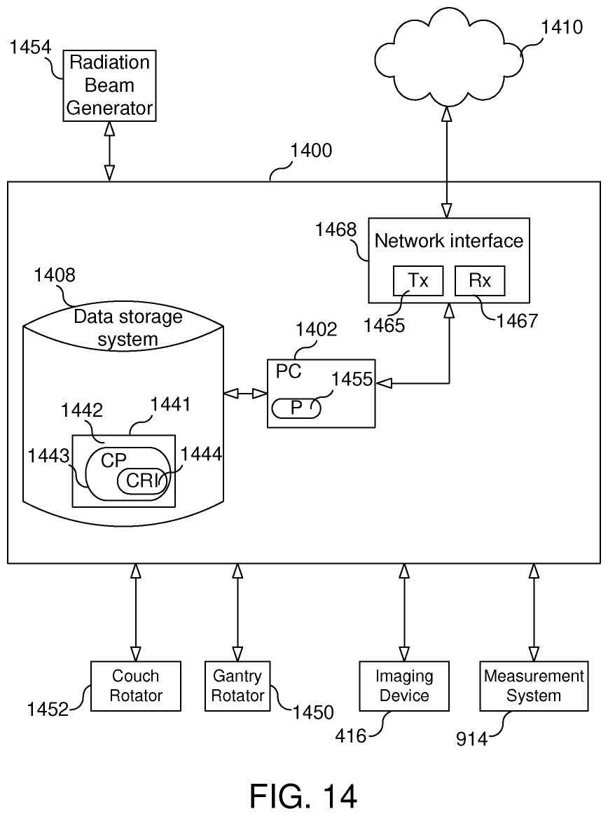

FIG. 14 is a block diagram of a controller 1400 of a LINAC 100 according to some aspects. As shown in FIG. 14, the controller 1400 may include: processing circuitry (PC) 1402, which may include one or more processors (P) 1455 (e.g., one or more general purpose microprocessors and/or one or more other processors, such as an application specific integrated circuit (ASIC), field-programmable gate arrays (FPGAs), and the like), which processors may be co-located in a single housing or in a single data center or may be geographically distributed (i.e., the system may be a distributed computing apparatus); a network interface 1468 including a transmitter (Tx) 1465 and a receiver (Rx) 1467 for enabling the controller 1400 to transmit data to and receive data from other nodes connected to a network 1410 (e.g., an Internet Protocol (IP) network) to which network interface 1468 is connected; and a local storage unit (a.k.a., “data storage system”) 1408, which may include one or more non-volatile storage devices and/or one or more volatile storage devices. In some aspects where PC 1402 includes a programmable processor, a computer program product (CPP) 1441 may be provided. In some aspects, the CPP 1441 may include a computer readable medium (CRM) 1442 storing a computer program (CP) 1443 comprising computer readable instructions (CRI) 1444. In some aspects, the CRM 1442 may be a non-transitory computer readable medium, such as, magnetic media (e.g., a hard disk), optical media, memory devices (e.g., random access memory, flash memory), and the like. In some aspects, the CRI 1444 of computer program 1443 may be configured such that when executed by PC 1402, the CRI causes the LINAC 100 to perform steps described herein (e.g., one or more steps described herein with reference to the flowcharts). In other aspects, the controller 1400 may be configured to perform steps described herein without the need for code. That is, for example, the PC 1402 may consist merely of one or more ASICs. Hence, the features of the aspects described herein may be implemented in hardware and/or software.

In some aspects, as shown in FIG. 14, the LINAC 100 may include a gantry rotator 1450 configured to rotate the gantry 102 about the gantry axis of rotation 208, and the controller 1400 may be configured to communicate with the gantry rotator 1450 (e.g., directly and/or via the network 1410). In some aspects, as shown in FIG. 14, the LINAC 100 may include a couch rotator 1452 configured to rotate the couch 106 about the couch axis of rotation 310, and the controller 1400 may be configured to communicate with the couch rotator 1452 (e.g., directly and/or via the network 1410). In some aspects, as shown in FIG. 14, the LINAC 100 may include a radiation beam generator 1454 configured to generate an electron beam in a waveguide of the LINAC 100, and the controller 1400 may be configured to communicate with the radiation beam generator 1454 (e.g., directly and/or via the network 1410). In some aspects, as shown in FIG. 14, the controller 1400 may be configured to communicate with the imaging device 416 of the LINAC 100 (e.g., directly and/or via the network 1410). In some aspects, as shown in FIG. 14, the controller 1400 may be configured to communicate with a measurement system 914 (e.g., directly and/or via the network 1410). In some aspects, the measurement system 914 may include, for example and without limitation, an optical tracking system, a kilovoltage imaging system, a megavoltage imaging system, surface imaging device, and/or a laser distance measurement device. In some aspects, some or all of the components (e.g., optical tracking system, kilovoltage imaging system, megavoltage imaging system, surface imaging device, and/or laser distance measurement device) may be part of (e.g., integrated in) in the LINAC 100. In some aspects, some or all of the components of the measurement system 914 may be external to and/or separate from the LINAC 100. In some aspects, as shown in FIG. 14, the measurement system 914 may be separate and distinct from the imaging device 416. However, this is not required, and, in some alternative aspects, the measurement system 914 may comprise or consist of the imaging device 416.

### 2.12 Apparatus

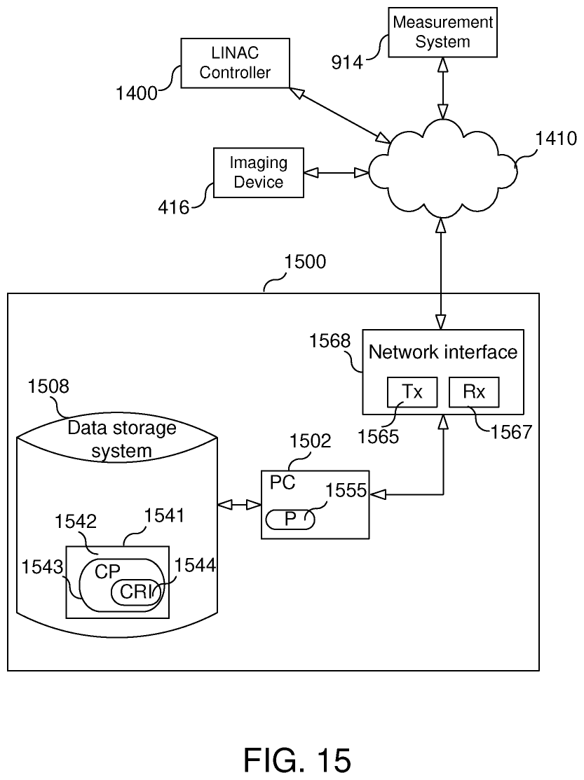

FIG. 15 is a block diagram of an apparatus 1500 according to some aspects. As shown in FIG. 15, the apparatus 1500 may include: processing circuitry (PC) 1502, which may include one or more processors (P) 1555 (e.g., one or more general purpose microprocessors and/or one or more other processors, such as an application specific integrated circuit (ASIC), field-programmable gate arrays (FPGAs), and the like), which processors may be co-located in a single housing or in a single data center or may be geographically distributed (i.e., the system may be a distributed computing apparatus); a network interface 1568 including a transmitter (Tx) 1565 and a receiver (Rx) 1567 for enabling the apparatus 1500 to transmit data to and receive data from other nodes connected to a network 1410 (e.g., an Internet Protocol (IP) network) to which network interface 1568 is connected; and a local storage unit (a.k.a., “data storage system”) 1508, which may include one or more non-volatile storage devices and/or one or more volatile storage devices. In some aspects where PC 1502 includes a programmable processor, a computer program product (CPP) 1541 may be provided. In some aspects, the CPP 1541 may include a computer readable medium (CRM) 1542 storing a computer program (CP) 1543 comprising computer readable instructions (CRI) 1544. In some aspects, the CRM 1542 may be a non-transitory computer readable medium, such as, magnetic media (e.g., a hard disk), optical media, memory devices (e.g., random access memory, flash memory), and the like. In some aspects, the CRI 1544 of computer program 1543 may be configured such that when executed by PC 1502, the CRI causes the apparatus 1500 to perform steps described herein (e.g., one or more steps described herein with reference to the flowcharts). In other aspects, the apparatus 1500 may be configured to perform steps described herein without the need for code. That is, for example, the PC 1502 may consist merely of one or more ASICs. Hence, the features of the aspects described herein may be implemented in hardware and/or software.

In some aspects, as shown in FIG. 15, the apparatus 1500 may be configured to communicate with the imaging device 416 of the LINAC 100 (e.g., directly and/or via the network 1410). In some aspects, as shown in FIG. 15, the apparatus 1500 may be configured to communicate with a measurement system 914 (e.g., directly and/or via the network 1410). In some aspects, the measurement system 914 may include, for example and without limitation, an optical tracking system, a kilovoltage imaging system, a megavoltage imaging system, surface imaging device, and/or a laser distance measurement device. In some aspects, as shown in FIG. 14, the measurement system 914 may be separate and distinct from the imaging device 416. However, this is not required, and, in some alternative aspects, the measurement system 914 may comprise or consist of the imaging device 416. In some aspects, as shown in FIG. 15, the apparatus 1500 may be configured to communicate with the controller 1400 of the LINAC 100 (e.g., directly and/or via the network 1410).

### 2.11 Systems

In some aspects, a system for QA in SIMT radiation therapy may include the LINAC 100, the phantom 900, the measurement system 914, and processing circuitry. In some aspects, the phantom 900 may include an off-isocenter radiation opaque marker 904, 906, or 908. In some aspects, the LINAC 100 may be configured to generate the radiation beam 412. In some aspects, the LINAC 100 may include the imaging device 416, which may be configured to acquire a two-dimensional (2D) radiation transmission image 1200 indicative of a radiation field of the generated radiation beam 412 after passing through the off-isocenter radiation opaque marker 904, 906, or 908 of the phantom 900, and the off-isocenter radiation opaque marker 904, 906, or 908 may be offset from an isocenter of the LINAC 100 at least one spatial dimension. In some aspects, the measurement system 914 may be configured to measure an actual marker position, which may be a measured position of the off-isocenter radiation opaque marker 904, 906, or 908 of the phantom 900, and the off-isocenter radiation opaque marker 904, 906, or 908 may be offset from an isocenter of the LINAC 100 at least one spatial dimension. In some aspects, the processing circuity of the system may include the PC 1402 of the LINAC 100 and/or the PC 1502 of the apparatus 1500. In some aspects, the processing circuitry may be configured to use the actual marker position to compute a beam's eye view marker position 1106, the beam's eye view marker position 1106 may be a projected position of the off-isocenter radiation opaque marker 904, 906, or 908 in an expected field contour 1102 of an expected radiation beam, and the expected radiation beam may be a radiation beam expected to be generated by the LINAC 100. In some aspects, the processing circuitry may be configured to determine a centroid 1304 of the expected field contour 1102. In some aspects, the processing circuitry may be configured to use the 2D radiation transmission image 1200 to determine (i) a measured field contour 1202 and (ii) a measured marker contour 1204. In some aspects, the measured field contour 1202 may be a boundary of the radiation field of the generated radiation beam 412 in the 2D radiation transmission image 1200, and the measured marker contour 1204 may be a boundary of a shadow of the off-isocenter radiation opaque marker 904, 906, or 908 in the radiation field of the generated radiation beam 412 in the 2D radiation transmission image 1200. In some aspects, the processing circuitry may be configured to determine a centroid 1306 of the measured field contour 1202 and a centroid of the measured marker contour 1204. In some aspects, the processing circuitry may be configured to align the beam's eye view marker position 1106 with the centroid of the measured marker contour 1204 at a common marker reference point 1302. In some aspects, the processing circuitry may be configured to, with the beam's eye view marker position 1106 aligned with the centroid of the measured marker contour 1204 at the common marker reference point 1302, determine a geometric error, and the geometric error may be a 2D distance between the centroid 1304 of the expected field contour 1102 and the centroid 1306 of the measured field contour 1202.

In some aspects, a system for QA in SIMT radiation therapy may include the phantom 900, the measurement system 914, and processing circuitry. In some aspects, the phantom 900 may include one or more off-isocenter radiation opaque markers 904, 906, and/or 908. In some aspects, the measurement system 914 may be configured to acquire megavoltage or kilovoltage images of the phantom 900, and the measurement system 914 may include a kilovoltage imaging system and/or a megavoltage imaging system. In some aspects, the processing circuitry may be configured to: (1) use the megavoltage or kilovoltage images to determine one or more three-dimensional (3D) positions of the one or more off-isocenter radiation opaque markers 904, 906, and/or 908 and (2) calculate a rotational deviation of the phantom 900. In some aspects, the measurement system 914 may be configured to acquire surface images of the phantom 900, and the measurement system 914 may include a surface imaging device. In some aspects, the processing circuitry may be configured to: (1) use the surface images to determine one or more 3D positions of the one or more off-isocenter radiation opaque markers 904, 906, and/or 908 and (2) calculate a rotational deviation of the phantom 900. In some aspects, calculating the rotational deviation of the phantom 900 may include comparing the one or more determined 3D positions of the one or more off-isocenter radiation opaque markers 904, 906, and/or 908 with one or more expected 3D positions of the one or more off-isocenter radiation opaque markers 904, 906, and/or 908.

In some aspects, a system for end-to-end QA in SIMT radiation therapy may include a phantom 900, a linear accelerator (LINAC) 100, and processing circuitry, In some aspects, the phantom 900 may include an off-isocenter radiation opaque marker 904, 906, or 908. In some aspects, the LINAC 100 may be configured to generate the radiation beam 412. In some aspects, the LINAC 100 may include the imaging device 416, which may be configured to acquire a two-dimensional (2D) radiation transmission image 1200 indicative of a radiation field of the generated radiation beam 412 after passing through the off-isocenter radiation opaque marker 904, 906, or 908 of the phantom 900, and the off-isocenter radiation opaque marker 904, 906, or 908 may be offset from an isocenter of the LINAC 100 at least one spatial dimension. In some aspects, the processing circuity of the system may include the PC 1402 of the LINAC 100 and/or the PC 1502 of the apparatus 1500. In some aspects, the processing circuitry may be configured to obtain a reference marker position from a treatment planning system (TPS). In some aspects, the reference marker position may be a derived position of the off-isocenter radiation opaque marker 904, 906, and/or 908 of the phantom 900. In some aspects, the reference marker position may be derived from one of: (i) a centroid of a marker structure contour defined in a radiotherapy (RT) structure set generated during treatment planning and (ii) a centroid of a volume of the off-isocenter radiation opaque marker 904, 906, and/or 908 as represented in an RT dose distribution computed by the TPS. In some aspects, the processing circuitry may be configured to project the reference marker position into a beam's-eye view corresponding to a planned gantry angle and couch angle of the LINAC, without applying a correction for physical positioning errors of the phantom 900. In some aspects, the processing circuitry may be configured to use the 2D radiation transmission image 1200 to determine (1) a measured field contour 1202 and (2) a measured marker contour 1204. The measured field contour 1202 may be a boundary of the radiation field of the generated radiation beam 412 in the 2D radiation transmission image 1200, and the measured marker contour 1204 may be a boundary of a shadow of the off-isocenter radiation opaque marker 904, 906, and/or 908 in the radiation field of the generated radiation beam 412 in the 2D radiation transmission image 1200. In some aspects, the processing circuitry may be configured to determine a centroid of the measured field contour 1202 and a centroid of the measured marker contour 1204. In some aspects, the processing circuitry may be configured to align the reference marker position with the centroid of the measured marker contour 1204 at a common marker reference point. In some aspects, the processing circuitry may be configured to, with the reference marker position aligned with the centroid of the measured marker contour 1204 at the common marker reference point, determine a geometric error that is a 2D distance between a centroid of an expected field contour of an expected radiation beam and the centroid of the measured field contour 1202. In some aspects, the expected radiation beam may be a radiation beam expected to be generated by the LINAC 100.

### 2.12 Flowcharts

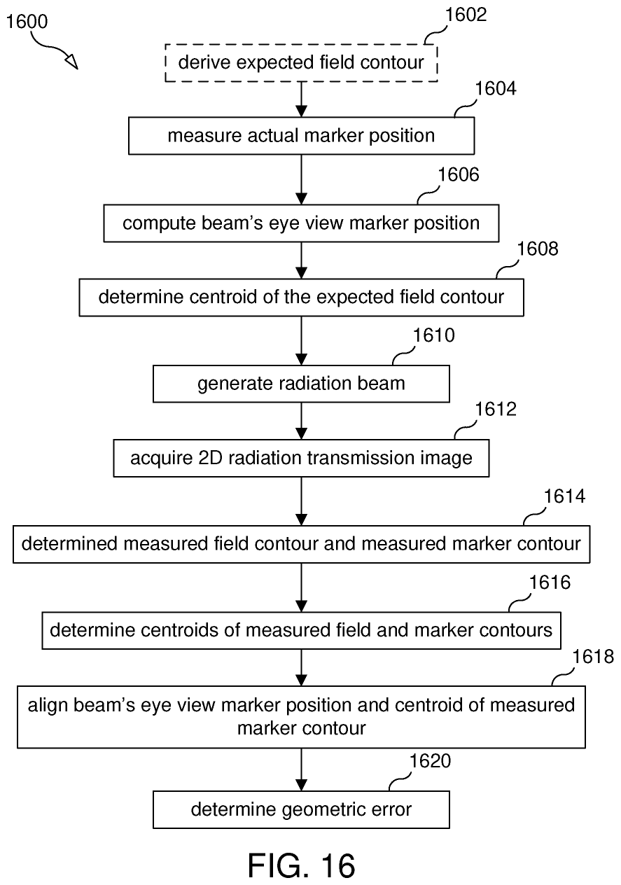

FIG. 16 illustrates a process 1600 for QA in SIMT radiation therapy according to some aspects. In some aspects, one or more of the steps of the process 1600 may be performed by the LINAC 100 (e.g., by the controller 1400 of the LINAC 100). In some aspects, one or more of the steps of the process 1600 may be additionally or alternatively be performed by an apparatus (e.g., the apparatus 1500 of FIG. 15).

In some aspects, as shown in FIG. 16, the process 1600 may include a step 1604 of using a measurement system 914 to measure an actual marker position in a 3D coordinate system of the LINAC 100. In some aspects, the actual marker position may be a measured position of an off-isocenter radiation opaque marker 904, 906, or 908 of a phantom 900, and the off-isocenter radiation opaque marker 904, 906, or 908 may be offset from an isocenter of the LINAC 100 at least one spatial dimension. In some aspects, using the measurement system 914 to measure the actual marker position in step 1604 may include using the measurement system 914 to measure the position and orientation of the phantom 900, and the mathematical transformation may include a rigid-body transformation encoding three translational and three rotational degrees of freedom representing the measured position and orientation of the phantom 900. In some aspects, the measurement system 914 may include an optical tracking system, a kilovoltage imaging system, a megavoltage imaging system, a surface imaging device, and/or a laser distance measurement device.

In some aspects, as shown in FIG. 16, the process 1600 may include a step 1606 of using the actual marker position to compute a beam's eye view marker position 1106, which may be a projected position of the off-isocenter radiation opaque marker 904, 906, or 908 in an expected field contour 1102 of an expected radiation beam, and the expected radiation beam may be a radiation beam expected to be generated by the LINAC 100. In some aspects, the beam's eye view marker position 1106 may not coincide with a center of the expected field contour 1102. In some aspects, computing the beam's eye view marker position 1106 in step 1606 may include applying a mathematical transformation to project the actual marker position into a beam's eye view of the expected field contour 1102. In some aspects, the beam's eye view may be a 2D plane perpendicular to a central axis of a radiation beam 412 generated by the LINAC 100. In some aspects, the beam's eye view marker position 1106 may be a projected position of the off-isocenter radiation opaque marker 904, 906, or 908 in the 2D plane that is perpendicular to the central axis of the radiation beam 412.

In some aspects, as shown in FIG. 16, the process 1600 may include a step 1608 of determining a centroid 1304 of the expected field contour 1102.

In some aspects, as shown in FIG. 16, the process 1600 may include a step 1610 of using the LINAC 100 to generate a radiation beam 412.

In some aspects, as shown in FIG. 16, the process 1600 may include a step 1612 of using an imaging device 416 of the LINAC 100 to acquire a two-dimensional (2D) radiation transmission image 1200 indicative of a radiation field of the generated radiation beam 412 after passing through the off-isocenter radiation opaque marker 904, 906, or 908 of the phantom 900. In some aspects, the imaging device 416 may be an electronic portal imaging device (EPID).

In some aspects, as shown in FIG. 16, the process 1600 may include a step 1614 of using the 2D radiation transmission image 1200 to determine (1) a measured field contour 1202 and (2) a measured marker contour 1204. In some aspects, the measured field contour 1202 may be a boundary of the radiation field of the generated radiation beam 412 in the 2D radiation transmission image 1200, and the measured marker contour 1204 may be a boundary of a shadow of the off-isocenter radiation opaque marker 904, 906, or 908 in the radiation field of the generated radiation beam 412 in the 2D radiation transmission image 1200.

In some aspects, as shown in FIG. 16, the process 1600 may include a step 1616 of determining a centroid 1306 of the measured field contour 1202 and a centroid of the measured marker contour 1204.

In some aspects, as shown in FIG. 16, the process 1600 may include a step 1618 of aligning the beam's eye view marker position 1106 with the centroid of the measured marker contour 1204 at a common marker reference point 1302.

In some aspects, as shown in FIG. 16, the process 1600 may include a step 1620 of, with the beam's eye view marker position 1106 aligned with the centroid of the measured marker contour 1204 at the common marker reference point 1302, determining a geometric error. In some aspects, the geometric error may be a 2D distance between the centroid 1304 of the expected field contour 1102 and the centroid 1306 of the measured field contour 1202. In some aspects, the process 1600 may further include performing a contour alignment comparison, computing a contour overlap metric, and/or performing a leaf-by-leaf multileaf collimator aperture comparison between the expected and measured field contours 1102 and 1202.

In some aspects, the process 1600 may further include using the geometric error determined in step 1620 to perform at least one of: approving a patient treatment if the geometric error is below a clinical acceptance threshold, adjusting planning target volume margins based on the geometric error, and limiting treatment to off-isocenter target locations whose geometric error satisfies the clinical acceptance threshold.

In some aspects, as shown in FIG. 16, the process 1600 may include an optional step 1602 of deriving the expected field contour 1102. In some aspects, the expected field contour 1102 may be derived in step 1602 based on one or both of: (1) geometric multileaf collimator (MLC) aperture and jaw settings specified in a radiotherapy plan file and projected into a beam's-eye view plane at a depth of the off-isocenter radiation opaque marker 904, 906, or 908; and (2) an isodose contour extracted from a 2D dose distribution computed by a treatment planning system (TPS) in the beam's-eye view plane at the depth of the off-isocenter radiation opaque marker 904, 906, or 908. In some aspects in which the expected field contour 1102 is derived in step 1602 based on the isodose contour extracted from the 2D dose distribution, the 2D dose distribution may be (i) extracted from a digitally-reconstructed radiograph created by the TPS, (ii) extracted from a 3D distribution using ray tracing techniques along the source to imaging panel directions, or (iii) extracted from one or more predicted portal dose maps derived from the TPS.

In some aspects, the steps 1606 through 1620 may be performed with respect to a first combination of gantry angle and couch angle, and the process 1600 may further include: repeating steps 1606 through 1620 for a plurality of different combinations of gantry angle and couch angle. In some aspects, the process 1600 may include aggregating the geometric errors determined at each combination of gantry angle and couch angle to determine an overall geometric accuracy of the LINAC 100 at the actual marker position.

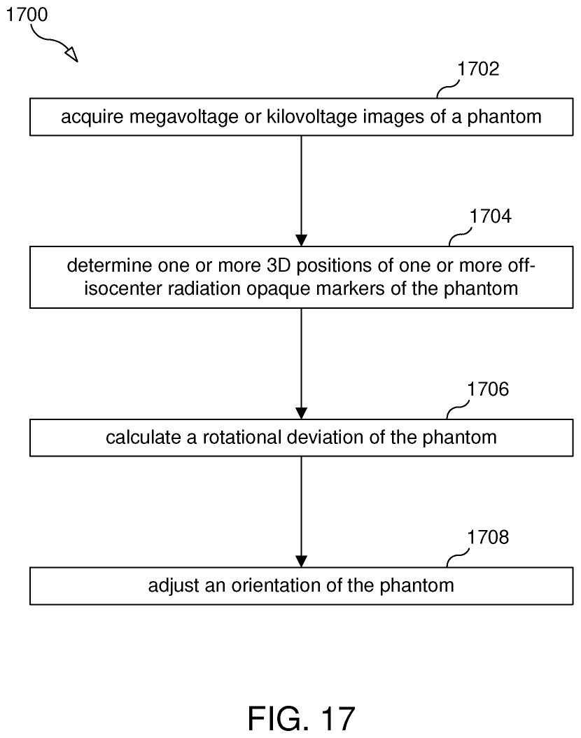

FIG. 17 illustrates a process 1700 for QA in SIMT radiation therapy according to some aspects. In some aspects, one or more of the steps of the process 1700 may be performed by the LINAC 100 (e.g., by the controller 1400 of the LINAC 100). In some aspects, one or more of the steps of the process 1700 may be additionally or alternatively be performed by an apparatus (e.g., the apparatus 1500 of FIG. 15).

In some aspects, as shown in FIG. 17, the process 1700 may include a step 1702 of using a measurement system 914 to acquire megavoltage or kilovoltage images of a phantom 900. In some aspects, the measurement system 914 may include a kilovoltage imaging system and/or a megavoltage imaging system. In some aspects, the phantom 900 may include one or more off-isocenter radiation opaque markers 904, 906, and/or 908.

In some aspects, as shown in FIG. 17, the process 1700 may include a step 1704 of using the megavoltage or kilovoltage images to determine one or more three-dimensional (3D) positions of the one or more off-isocenter radiation opaque markers 904, 906, and/or 908. In some aspects, the 3D positions of the one or more off-isocenter radiation opaque markers 904, 906, and/or 908 may be determined in step 1704 by acquiring the megavoltage or kilovoltage images at two or more gantry angles and reconstructing 3D coordinates of the one or more off-isocenter radiation opaque markers 904, 906, and/or 908 through geometric back-projection.

In some aspects, as shown in FIG. 17, the process 1700 may include a step 1706 of calculating a rotational deviation of the phantom 900. In some aspects, calculating the rotational deviation of the phantom 900 may include comparing the one or more determined 3D positions of the one or more off-isocenter radiation opaque markers 904, 906, and/or 908 with one or more expected 3D positions of the one or more off-isocenter radiation opaque markers 904, 906, and/or 908.

In some aspects, as shown in FIG. 17, the process 1700 may include a step 1708 of using the calculated rotational deviation of the phantom 900 to adjust an orientation of the phantom 900. In some aspects, adjusting the orientation of the phantom 900 in step 1708 may include physically adjusting the orientation of the phantom 900 to reduce the rotational deviation of the phantom 900. In some aspects, adjusting the orientation of the phantom 900 in step 1708 may additionally or alternatively include mathematically adjusting the orientation of the phantom 900 in an analysis of delivered radiation fields. In some aspects, adjusting the orientation of the phantom 900 in step 1708 may additionally or alternatively include computing one or more corrected 3D positions for each of the one or more off-isocenter radiation opaque markers 904, 906, and/or 908 by applying a rigid-body transformation encoding the calculated rotational deviation of the phantom 900 to the one or more expected 3D positions of the one or more off-isocenter radiation opaque markers 904, 906, and/or 908.

In some aspects, the process 1700 may further include (1) for each off-isocenter radiation opaque marker of the one or more off-isocenter radiation opaque markers 904, 906, and/or 908, using the actual marker position to compute a beam's eye view marker position 1106. In some aspects, the beam's eye view marker position 1106 may be a projected position of the off-isocenter radiation opaque marker in an expected field contour 1102 of an expected radiation beam associated with the off-isocenter radiation opaque marker, and the expected radiation beam may be a radiation beam expected to be generated by the LINAC 100. In some aspects, for each off-isocenter radiation opaque marker of the one or more off-isocenter radiation opaque markers 904, 906, and/or 908, computing the beam's eye view marker position 1106 may include applying a mathematical transformation to project the actual marker position into a beam's eye view of the expected field contour 1102. In some aspects, the beam's eye view may be a 2D plane perpendicular to a central axis of a radiation beam 412 generated by the LINAC 100. In some aspects, the beam's eye view marker position 1106 may be a projected position of the off-isocenter radiation opaque marker 904, 906, or 908 in the 2D plane that is perpendicular to the central axis of the radiation beam 412. In some aspects, the process 1700 may further include (2) for each off-isocenter radiation opaque marker of the one or more off-isocenter radiation opaque markers 904, 906, and/or 908, determining a centroid 1304 of the expected field contour 1102. In some aspects, the process 1700 may further include (3) for each off-isocenter radiation opaque marker of the one or more off-isocenter radiation opaque markers 904, 906, and/or 908, using the LINAC 100 to generate a radiation beam 412. In some aspects, the process 1700 may further include (4) for each off-isocenter radiation opaque marker of the one or more off-isocenter radiation opaque markers 904, 906, and/or 908, using an imaging device 416 of the LINAC 100 to acquire a two-dimensional (2D) radiation transmission image 1200 indicative of a radiation field of the generated radiation beam 412 after passing through the off-isocenter radiation opaque marker of the phantom 900. In some aspects, the process 1700 may further include (5) for each off-isocenter radiation opaque marker of the one or more off-isocenter radiation opaque markers 904, 906, and/or 908, using the 2D radiation transmission image 1200 to determine (i) a measured field contour 1202 and (ii) measured marker contour 1204. In some aspects, the measured field contour 1202 may be a boundary of the radiation field of the generated radiation beam 412 in the 2D radiation transmission image 1200, and the measured marker contour 1204 may be a boundary of a shadow of the off-isocenter radiation opaque marker in the radiation field of the generated radiation beam 412 in the 2D radiation transmission image 1200. In some aspects, the process 1700 may further include (6) for each off-isocenter radiation opaque marker of the one or more off-isocenter radiation opaque markers 904, 906, and/or 908, determining a centroid 1306 of the measured field contour 1202 and a centroid of the measured marker contour 1204. In some aspects, the process 1700 may further include (7) for each off-isocenter radiation opaque marker of the one or more off-isocenter radiation opaque markers 904, 906, and/or 908, aligning the beam's eye view marker position 1106 with the centroid of the measured marker contour 1204 at a common marker reference point 1302. In some aspects, the process 1700 may further include (8) for each off-isocenter radiation opaque marker of the one or more off-isocenter radiation opaque markers 904, 906, and/or 908, with the beam's eye view marker position 1106 aligned with the centroid of the measured marker contour 1204 at the common marker reference point 1302, determining a geometric error, which may be a 2D distance between the centroid 1304 of the expected field contour 1102 and the centroid 1306 of the measured field contour 1202.

In some aspects, the step 1702 may additionally or alternatively include using a measurement system 914 to acquire surface images of the phantom 900. In some aspects, the step 1704 may additionally or alternatively include using the surface images to determine 3D positions of the one or more off-isocenter radiation opaque markers 904, 906, and/or 908. In some aspects, the 3D positions of the one or more off-isocenter radiation opaque markers 904, 906, and/or 908 may be determined in step 1704 by acquiring the surface images at two or more gantry angles and reconstructing 3D coordinates of the one or more off-isocenter radiation opaque markers 904, 906, and/or 908 through geometric back-projection.

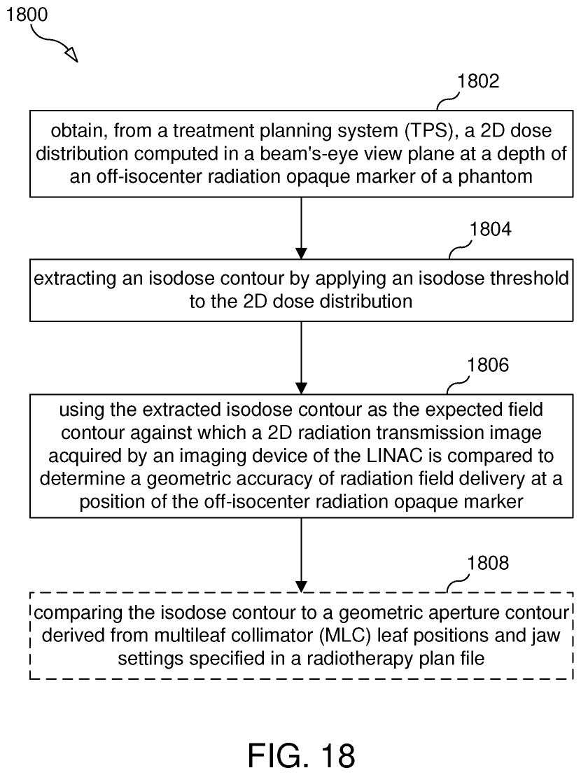

FIG. 18 illustrates a process 1800 for generating an expected field contour 1102 for use in QA of SIMT radiation therapy treatments according to some aspects. In some aspects, one or more of the steps of the process 1800 may be performed by the LINAC 100 (e.g., by the controller 1400 of the LINAC 100). In some aspects, one or more of the steps of the process 1800 may be additionally or alternatively be performed by an apparatus (e.g., the apparatus 1500 of FIG. 15).

In some aspects, as shown in FIG. 18, the process 1800 may include a step 1802 of obtaining, from a treatment planning system (TPS), a two-dimensional (2D) dose distribution computed in a beam's-eye view plane at a depth of an off-isocenter radiation opaque marker 904, 906, or 908 of a phantom 900. In some aspects, the off-isocenter radiation opaque marker 904, 906, and/or 908 may be offset from an isocenter of a LINAC 100 in at least one spatial dimension. In some aspects, the 2D dose distribution may be (i) extracted from a digitally-reconstructed radiograph created by the TPS, (ii) extracted from a three-dimensional (3D) distribution using ray tracing techniques along the source to imaging panel directions, or (iii) extracted from one or more predicted portal dose maps derived from the TPS.

In some aspects, as shown in FIG. 18, the process 1800 may include a step 1804 of extracting an isodose contour by applying an isodose threshold to the 2D dose distribution. In some aspects, the isodose threshold may be a 50% isodose level normalized to one of: a prescription dose, a field maximum dose, and a user-specified reference dose. However, this is not required, and, in some alternative aspects, a different isodose threshold may be used (e.g., any threshold within a range of 45-55% isodose level, any threshold within a range of 40-60% isodose level, or any threshold within a range of 30-70% isodose level).

In some aspects, as shown in FIG. 18, the process 1800 may include a step 1806 of using the extracted isodose contour as the expected field contour 1102 against which a 2D radiation transmission image 1200 acquired by an imaging device 416 of the LINAC 100 is compared to determine a geometric accuracy of radiation field delivery at a position of the off-isocenter radiation opaque marker 904, 906, and/or 908.

In some aspects, as shown in FIG. 18, the process 1800 may include an optional step 1808 of comparing the isodose contour to a geometric aperture contour derived from multileaf collimator (MLC) leaf positions and jaw settings specified in a radiotherapy plan file. In some aspects, the geometric aperture contour may be used to assess MLC positioning accuracy, and the isodose contour may be used to assess dosimetric field accuracy at the position of the off-isocenter radiation opaque marker 904, 906, and/or 908.

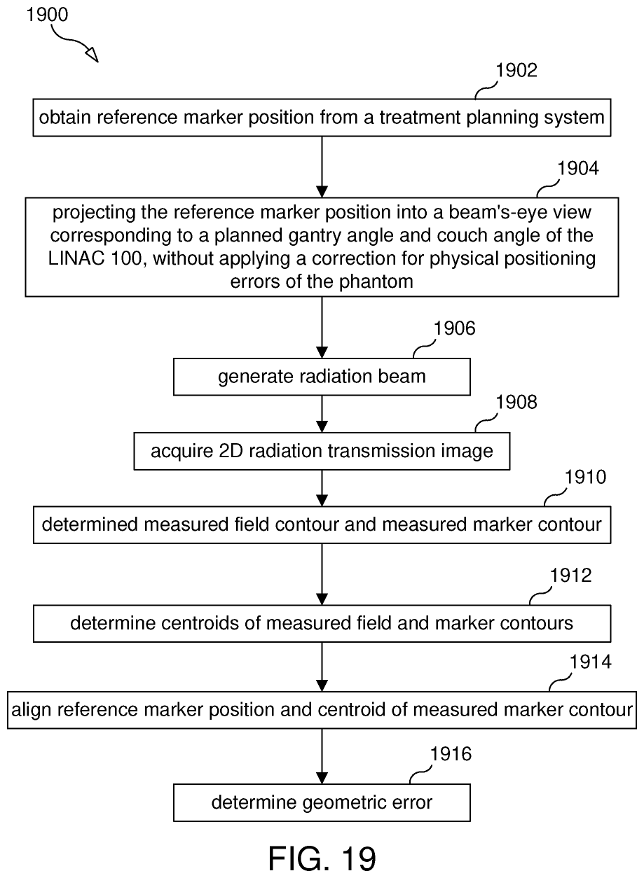

FIG. 19 illustrates a process 1900 for end-to-end quality assurance in single-isocenter, multi-target (SIMT) radiation therapy according to some aspects. In some aspects, one or more of the steps of the process 1900 may be performed by the LINAC 100 (e.g., by the controller 1400 of the LINAC 100). In some aspects, one or more of the steps of the process 1900 may be additionally or alternatively be performed by an apparatus (e.g., the apparatus 1500 of FIG. 15).

In some aspects, as shown in FIG. 19, the process 1900 may include a step 1902 of obtaining a reference marker position from a treatment planning system (TPS). In some aspects, the reference marker position may be a derived position of an off-isocenter radiation opaque marker 904, 906, and/or 908 of a phantom 900, and the off-isocenter radiation opaque marker 904, 906, and/or 908 may be offset from an isocenter of a linear accelerator (LINAC) 100 in at least one spatial dimension. In some aspects, the reference marker position may be derived from one of: (i) a centroid of a marker structure contour defined in a radiotherapy (RT) structure set generated during treatment planning and (ii) a centroid of a volume of the off-isocenter radiation opaque marker 904, 906, and/or 908 as represented in an RT dose distribution computed by the TPS.

In some aspects, as shown in FIG. 19, the process 1900 may include a step 1904 of projecting the reference marker position into a beam's-eye view corresponding to a planned gantry angle and couch angle of the LINAC 100, without applying a correction for physical positioning errors of the phantom 900.

In some aspects, as shown in FIG. 19, the process 1900 may include a step 1906 of using the LINAC 100 to generate a radiation beam 412.

In some aspects, as shown in FIG. 19, the process 1900 may include a step 1908 of using an imaging device 416 of the LINAC 100 to acquire a two-dimensional (2D) radiation transmission image 1200 indicative of a radiation field of the generated radiation beam 412 after passing through the off-isocenter radiation opaque marker 904, 906, and/or 908 of the phantom 900.

In some aspects, as shown in FIG. 19, the process 1900 may include a step 1910 of using the 2D radiation transmission image 1200 to determine (1) a measured field contour 1202 and (2) a measured marker contour 1204. The measured field contour 1202 may be a boundary of the radiation field of the generated radiation beam 412 in the 2D radiation transmission image 1200, and the measured marker contour 1204 may be a boundary of a shadow of the off-isocenter radiation opaque marker 904, 906, and/or 908 in the radiation field of the generated radiation beam 412 in the 2D radiation transmission image 1200.

In some aspects, as shown in FIG. 19, the process 1900 may include a step 1912 of determining a centroid of the measured field contour 1202 and a centroid of the measured marker contour 1204.

In some aspects, as shown in FIG. 19, the process 1900 may include a step 1914 of aligning the reference marker position with the centroid of the measured marker contour 1204 at a common marker reference point.

In some aspects, as shown in FIG. 19, the process 1900 may include a step 1916 of with the reference marker position aligned with the centroid of the measured marker contour 1204 at the common marker reference point, determining a geometric error. In some aspects, the geometric error may be a 2D distance between a centroid of an expected field contour 1102 of an expected radiation beam and the centroid of the measured field contour 1202, and the expected radiation beam may be a radiation beam expected to be generated by the LINAC 100.

In some aspects, the reference marker position may be derived from the centroid of the marker structure contour in the RT structure set, and the RT structure set may have been generated from a planning computed tomography image of the phantom 900 acquired prior to the quality assurance session.

In some aspects, the reference marker position may be derived from the centroid of the volume of the off-isocenter radiation opaque marker 904, 906, and/or 908 in the RT dose distribution computed by the TPS, and the RT dose distribution may have been computed using a beam geometry and multileaf collimator (MLC) aperture sequences that are the same as a beam geometry and MLC aperture sequences used for the generated radiation field.

In some aspects, steps (a) through (h) may be performed with respect to a first combination of gantry angle and couch angle, and the process 1900 may further include repeating steps (a) through (h) for a plurality of different combinations of gantry angle and couch angle. In some aspects, the process 1900 may further include determining geometric errors using an orientation-corrected method for quality assurance in SIMT radiation therapy (e.g., the process 1600 described above with respect to FIG. 16) on a 2D radiation transmission image dataset that is the same as a 2D radiation transmission image dataset used in the method for end-to-end quality assurance in SIMT radiation therapy. In some aspects, the process 1900 may further include comparing the geometric errors produced by the method for end-to-end quality assurance in SIMT radiation therapy with the geometric errors produced by the orientation-corrected method. In some aspects, the process 1900 may further include attributing differences between the geometric errors to phantom setup error, thereby decomposing the total end-to-end system error into its LINAC geometric error component and its phantom setup error component.

While various embodiments are described herein, it should be understood that they have been presented by way of example only, and not limitation. Thus, the breadth and scope of this disclosure should not be limited by any of the above-described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

What is claimed is:

1. A method for quality assurance in single-isocenter, multi-target (SIMT) radiation therapy, the method comprising:
(a) using a measurement system to measure an actual marker position in a three-dimensional (3D) coordinate system of a linear accelerator (LINAC), wherein the actual marker position is a measured position of an off-isocenter radiation opaque marker of a phantom, and the off-isocenter radiation opaque marker is offset from an isocenter of the LINAC in at least one spatial dimension;
(b) using the actual marker position to compute a beam's eye view marker position, wherein the beam's eye view marker position is a projected position of the off-isocenter radiation opaque marker in an expected field contour of an expected radiation beam, and the expected radiation beam is a radiation beam expected to be generated by the LINAC;
(c) determining a centroid of the expected field contour;
(d) using the LINAC to generate a radiation beam;
(e) using an imaging device of the LINAC to acquire a two-dimensional (2D) radiation transmission image indicative of a radiation field of the generated radiation beam after passing through the off-isocenter radiation opaque marker of the phantom;
(f) using the 2D radiation transmission image to determine (1) a measured field contour and (2) a measured marker contour, wherein the measured field contour is a boundary of the radiation field of the generated radiation beam in the 2D radiation transmission image, and the measured marker contour is a boundary of a shadow of the off-isocenter radiation opaque marker in the radiation field of the generated radiation beam in the 2D radiation transmission image;
(g) determining a centroid of the measured field contour and a centroid of the measured marker contour;
(h) aligning the beam's eye view marker position with the centroid of the measured marker contour at a common marker reference point; and
(i) with the beam's eye view marker position aligned with the centroid of the measured marker contour at the common marker reference point, determining a geometric error, wherein the geometric error is a 2D distance between the centroid of the expected field contour and the centroid of the measured field contour.

2. The method of claim 1, wherein the beam's eye view marker position does not coincide with a center of the expected field contour.

3. The method of claim 1, further comprising deriving the expected field contour.

4. The method of claim 3, wherein the expected field contour is derived based on one or both of:
(1) geometric multileaf collimator (MLC) aperture and jaw settings specified in a radiotherapy plan file and projected into a beam's-eye view plane at a depth of the off-isocenter radiation opaque marker; and
(2) an isodose contour extracted from a 2D dose distribution computed by a treatment planning system in the beam's-eye view plane at the depth of the off-isocenter radiation opaque marker.

5. The method of claim 1, wherein using the measurement system to measure the actual marker position comprises using the measurement system to measure the position and orientation of the phantom, and computing the beam's eye view marker position comprises applying a rigid-body transformation encoding three translational and three rotational degrees of freedom representing the measured position and orientation of the phantom to project the actual marker position into the beam's eye view of the expected field contour.

6. The method of claim 1, further comprising performing a contour alignment comparison, computing a contour overlap metric, and/or performing a leaf-by-leaf multileaf collimator aperture comparison between the expected and measured field contours.

7. The method of claim 1, wherein the measurement system comprises an optical tracking system, a kilovoltage imaging system, a megavoltage imaging system, a surface imaging device, and/or a laser distance measurement device.

8. The method of claim 1, wherein the imaging device is an electronic portal imaging device (EPID).

9. The method of claim 1, wherein steps (b) through (i) are performed with respect to a first combination of gantry angle and couch angle, and the method further comprises:
repeating steps (b) through (i) for a plurality of different combinations of gantry angle and couch angle; and
aggregating the geometric errors determined at each combination of gantry angle and couch angle to determine an overall geometric accuracy of the LINAC at the actual marker position.

10. The method of claim 1, further comprising using the determined geometric error to perform at least one of:
approving a patient treatment if the geometric error is below a clinical acceptance threshold;
adjusting planning target volume margins based on the geometric error; and
limiting treatment to off-isocenter target locations whose geometric error satisfies the clinical acceptance threshold.

11. A system for quality assurance in single-isocenter, multi-target (SIMT) radiation therapy, the system comprising:
(1) a phantom comprising an off-isocenter radiation opaque marker;
(2) a linear accelerator (LINAC) configured to generate a radiation beam, wherein the LINAC comprises an imaging device configured to acquire a two-dimensional (2D) radiation transmission image indicative of a radiation field of the generated radiation beam after passing through the off-isocenter radiation opaque marker of the phantom, and the off-isocenter radiation opaque marker is offset from an isocenter of the LINAC in at least one spatial dimension;
(3) a measurement system configured to measure an actual marker position in a three-dimensional (3D) coordinate system of the LINAC, wherein the actual marker position is a measured position of the off-isocenter radiation opaque marker of the phantom, and the off-isocenter radiation opaque marker is offset from an isocenter of the LINAC in at least one spatial dimension; and
(4) processing circuitry configured to:
(a) use the actual marker position to compute a beam's eye view marker position, wherein the beam's eye view marker position is a projected position of the off-isocenter radiation opaque marker in an expected field contour of an expected radiation beam, and the expected radiation beam is a radiation beam expected to be generated by the LINAC;
(b) determine a centroid of the expected field contour;
(c) use the 2D radiation transmission image to determine (i) a measured field contour and (ii) a measured marker contour, wherein the measured field contour is a boundary of the radiation field of the generated radiation beam in the 2D radiation transmission image, and the measured marker contour is a boundary of a shadow of the off-isocenter radiation opaque marker in the radiation field of the generated radiation beam in the 2D radiation transmission image;
(d) determine a centroid of the measured field contour and a centroid of the measured marker contour;
(e) align the beam's eye view marker position with the centroid of the measured marker contour at a common marker reference point; and
(f) with the beam's eye view marker position aligned with the centroid of the measured marker contour at the common marker reference point, determine a geometric error, wherein the geometric error is a 2D distance between the centroid of the expected field contour and the centroid of the measured field contour.

12. A method for quality assurance in single-isocenter, multi-target (SIMT) radiation therapy, the method comprising:
(a) using a measurement system to acquire megavoltage, kilovoltage, and/or surface images of a phantom, wherein the measurement system comprises a kilovoltage imaging system, a megavoltage imaging system, and/or a surface imaging device, and the phantom comprises one or more off-isocenter radiation opaque markers;
(b) using the megavoltage, kilovoltage, and/or surface images to determine one or more three-dimensional (3D) positions of the one or more off-isocenter radiation opaque markers;
(c) calculating a rotational deviation of the phantom, wherein calculating the rotational deviation of the phantom comprises comparing the one or more determined 3D positions of the one or more off-isocenter radiation opaque markers with one or more expected 3D positions of the one or more off-isocenter radiation opaque markers; and
(d) using the calculated rotational deviation of the phantom to adjust an orientation of the phantom.

13. The method of claim 12, wherein adjusting the orientation of the phantom comprises physically adjusting the orientation of the phantom to reduce the rotational deviation of the phantom.

14. The method of claim 12, wherein adjusting the orientation of the phantom comprises mathematically adjusting the orientation of the phantom in an analysis of delivered radiation fields.

15. The method of claim 12, wherein adjusting the orientation of the phantom comprises computing one or more corrected 3D positions for each of the one or more off-isocenter radiation opaque markers by applying a rigid-body transformation encoding the calculated rotational deviation of the phantom to the one or more expected 3D positions of the one or more off-isocenter radiation opaque markers.

16. The method of claim 12, wherein the 3D positions of the one or more off-isocenter radiation opaque markers are determined by acquiring the megavoltage, kilovoltage, and/or surface images at two or more gantry angles and reconstructing 3D coordinates of the one or more off-isocenter radiation opaque markers through geometric back-projection.

17. The method of claim 12, further comprising, for each off-isocenter radiation opaque marker of the one or more off-isocenter radiation opaque markers:
(1) using the actual marker position to compute a beam's eye view marker position, wherein the beam's eye view marker position is a projected position of the off-isocenter radiation opaque marker in an expected field contour of an expected radiation beam associated with the off-isocenter radiation opaque marker, and the expected radiation beam is a radiation beam expected to be generated by the LINAC;
(2) determining a centroid of the expected field contour;
(3) using the LINAC to generate a radiation beam;
(4) using an imaging device of the LINAC to acquire a two-dimensional (2D) radiation transmission image indicative of a radiation field of the generated radiation beam after passing through the off-isocenter radiation opaque marker of the phantom;
(5) using the 2D radiation transmission image to determine (i) a measured field contour and (ii) measured marker contour, wherein the measured field contour is a boundary of the radiation field of the generated radiation beam in the 2D radiation transmission image, and the measured marker contour is a boundary of a shadow of the off-isocenter radiation opaque marker in the radiation field of the generated radiation beam in the 2D radiation transmission image;
(6) determining a centroid of the measured field contour and a centroid of the measured marker contour;
(7) aligning the beam's eye view marker position with the centroid of the measured marker contour at a common marker reference point; and
(8) with the beam's eye view marker position aligned with the centroid of the measured marker contour at the common marker reference point, determining a geometric error, wherein the geometric error is a 2D distance between the centroid of the expected field contour and the centroid of the measured field contour.

18. The method of claim 17, wherein, for each off-isocenter radiation opaque marker of the one or more off-isocenter radiation opaque markers, computing the beam's eye view marker position comprises applying a mathematical transformation to project the actual marker position into a beam's eye view of the expected field contour.

19. A system for quality assurance in single-isocenter, multi-target (SIMT) radiation therapy, the system comprising:
a phantom comprising one or more off-isocenter radiation opaque markers;
a measurement system configured to acquire megavoltage, kilovoltage, and/or surface images of a phantom, wherein the measurement system comprises a kilovoltage imaging system, a megavoltage imaging system, and/or a surface imaging device;
processing circuitry configured to:
(1) use the megavoltage, kilovoltage, and/or surface images to determine one or more three-dimensional (3D) positions of the one or more off-isocenter radiation opaque markers; and
(2) calculate a rotational deviation of the phantom, wherein calculating the rotational deviation of the phantom comprises comparing the one or more determined 3D positions of the one or more off-isocenter radiation opaque markers with one or more expected 3D positions of the one or more off-isocenter radiation opaque markers.

20. A method for generating an expected field contour for use in quality assurance of single-isocenter, multi-target (SIMT) radiation therapy treatments, the method comprising:
(a) obtaining, from a treatment planning system (TPS), a two-dimensional (2D) dose distribution computed in a beam's-eye view plane at a depth of an off-isocenter radiation opaque marker of a phantom, wherein the off-isocenter radiation opaque marker is offset from an isocenter of a linear accelerator (LINAC) in at least one spatial dimension;
(b) extracting an isodose contour by applying an isodose threshold to the 2D dose distribution; and
(c) using the extracted isodose contour as the expected field contour against which a 2D radiation transmission image acquired by an imaging device of the LINAC is compared to determine a geometric accuracy of radiation field delivery at a position of the off-isocenter radiation opaque marker.

21. The method of claim 20, wherein the isodose threshold is a 50% isodose level normalized to one of: a prescription dose, a field maximum dose, and a user-specified reference dose.

22. The method of claim 20, further comprising comparing the isodose contour to a geometric aperture contour derived from multileaf collimator (MLC) leaf positions and jaw settings specified in a radiotherapy plan file, wherein the geometric aperture contour is used to assess MLC positioning accuracy, and the isodose contour is used to assess dosimetric field accuracy at the position of the off-isocenter radiation opaque marker.

23. The method of claim 20, wherein the 2D dose distribution is (i) extracted from a digitally-reconstructed radiograph created by the TPS, (ii) extracted from a three-dimensional (3D) distribution using ray tracing techniques along the source to imaging panel directions, or (iii) extracted from one or more predicted portal dose maps derived from the TPS.

24. A method for end-to-end quality assurance in single-isocenter, multi-target (SIMT) radiation therapy, the method comprising:
(a) obtaining a reference marker position from a treatment planning system (TPS), wherein the reference marker position is a derived position of an off-isocenter radiation opaque marker of a phantom, the off-isocenter radiation opaque marker is offset from an isocenter of a linear accelerator (LINAC) in at least one spatial dimension, and the reference marker position is derived from one of: (i) a centroid of a marker structure contour defined in a radiotherapy (RT) structure set generated during treatment planning and (ii) a centroid of a volume of the off-isocenter radiation opaque marker as represented in an RT dose distribution computed by the TPS;
(b) projecting the reference marker position into a beam's-eye view corresponding to a planned gantry angle and couch angle of the LINAC, without applying a correction for physical positioning errors of the phantom;
(c) using the LINAC to generate a radiation beam;
(d) using an imaging device of the LINAC to acquire a two-dimensional (2D) radiation transmission image indicative of a radiation field of the generated radiation beam after passing through the off-isocenter radiation opaque marker of the phantom;
(e) using the 2D radiation transmission image to determine (1) a measured field contour and (2) a measured marker contour, wherein the measured field contour is a boundary of the radiation field of the generated radiation beam in the 2D radiation transmission image, and the measured marker contour is a boundary of a shadow of the off-isocenter radiation opaque marker in the radiation field of the generated radiation beam in the 2D radiation transmission image;
(f) determining a centroid of the measured field contour and a centroid of the measured marker contour;
(g) aligning the reference marker position with the centroid of the measured marker contour at a common marker reference point; and
(h) with the reference marker position aligned with the centroid of the measured marker contour at the common marker reference point, determining a geometric error, wherein the geometric error is a 2D distance between a centroid of an expected field contour of an expected radiation beam and the centroid of the measured field contour, and the expected radiation beam is a radiation beam expected to be generated by the LINAC.

25. The method of claim 24, wherein the reference marker position is derived from the centroid of the marker structure contour in the RT structure set, and the RT structure set was generated from a planning computed tomography image of the phantom acquired prior to the quality assurance session.

26. The method of claim 24, wherein the reference marker position is derived from the centroid of the volume of the off-isocenter radiation opaque marker in the RT dose distribution computed by the TPS, and the RT dose distribution was computed using a beam geometry and multileaf collimator (MLC) aperture sequences that are the same as a beam geometry and MLC aperture sequences used for the generated radiation field.

27. The method of claim 24, wherein steps (a) through (h) are performed with respect to a first combination of gantry angle and couch angle, and the method further comprises repeating steps (a) through (h) for a plurality of different combinations of gantry angle and couch angle.

28. The method of claim 27, further comprising:
determining geometric errors using an orientation-corrected method for quality assurance in SIMT radiation therapy on a 2D radiation transmission image dataset that is the same as a 2D radiation transmission image dataset used in the method for end-to-end quality assurance in SIMT radiation therapy;
comparing the geometric errors produced by the method for end-to-end quality assurance in SIMT radiation therapy with the geometric errors produced by the orientation-corrected method; and
attributing differences between the geometric errors to phantom setup error, thereby decomposing the total end-to-end system error into its LINAC geometric error component and its phantom setup error component.

29. A system for end-to-end quality assurance in single-isocenter, multi-target (SIMT) radiation therapy, the system comprising:
(1) a phantom comprising an off-isocenter radiation opaque marker;
(2) a linear accelerator (LINAC) configured to generate a radiation beam, wherein the LINAC comprises an imaging device configured to acquire a two-dimensional (2D) radiation transmission image indicative of a radiation field of the generated radiation beam after passing through the off-isocenter radiation opaque marker of the phantom, and the off-isocenter radiation opaque marker is offset from an isocenter of the LINAC in at least one spatial dimension; and
(3) processing circuitry configured to:
(a) obtain a reference marker position from a treatment planning system (TPS), wherein the reference marker position is a derived position of the off-isocenter radiation opaque marker of the phantom, and the reference marker position is derived from one of: (i) a centroid of a marker structure contour defined in a radiotherapy (RT) structure set generated during treatment planning and (ii) a centroid of a volume of the off-isocenter radiation opaque marker as represented in an RT dose distribution computed by the TPS;
(b) project the reference marker position into a beam's-eye view corresponding to a planned gantry angle and couch angle of the LINAC, without applying a correction for physical positioning errors of the phantom;
(c) use the 2D radiation transmission image to determine (1) a measured field contour and (2) a measured marker contour, wherein the measured field contour is a boundary of the radiation field of the generated radiation beam in the 2D radiation transmission image, and the measured marker contour is a boundary of a shadow of the off-isocenter radiation opaque marker in the radiation field of the generated radiation beam in the 2D radiation transmission image;
(d) determine a centroid of the measured field contour and a centroid of the measured marker contour;
(e) align the reference marker position with the centroid of the measured marker contour at a common marker reference point; and
(f) with the reference marker position aligned with the centroid of the measured marker contour at the common marker reference point, determine a geometric error, wherein the geometric error is a 2D distance between a centroid of an expected field contour of an expected radiation beam and the centroid of the measured field contour, and the expected radiation beam is a radiation beam expected to be generated by the LINAC.
